SEQUENCE LISTING (13 sequences)

SEQ ID NO: 1 - DNA, 1522 bp, Homo sapiens
  CDS (13)..(1389)
  mat_peptide (124)..(1389)
acagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 2 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 3 - DNA, 540 bp, Homo sapiens
  promoter (1)..(540)
cttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacag

SEQ ID NO: 4 - DNA, 10898 bp, Homo sapiens
  gene (1)..(10898)
  misc_feature (1974)..(1974): n is a, c, g, or t
ggatcctgttgactcgtgaccttacccccaaccctgtgctctctgaaacatgagctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgaccttccctccactattgtccatgaccctgccaaatccccctctgtgagaaacacccaagaattatcaataaaaaaataaatttaaaaaaaaaatacaaaaaaaaaaaaaaaaaaaaaaaaagacttacgaatagttattgataaatgaatagctattggtaaagccaagtaaatgatcatattcaaaaccagacggccatcatcacagctcaagtctacctgatttgatctctttatcattgtcattctttggattcactagattagtcatcatcctcaaaattctcccccaagttctaattacgttccaaacatttaggggttacatgaagcttgaacctactaccttctttgcttttgagccatgagttgtaggaatgatgagtttacaccttacatgctggggattaatttaaactttacctctaagtcagttgggtagcctttggcttatttttgtagctaattttgtagttaatggatgcactgtgaatcttgctatgatagttttcctccacactttgccactaggggtaggtaggtactcagttttcagtaattgcttacctaagaccctaagccctatttctcttgtactggcctttatctgtaatatgggcatatttaatacaatataatttttggagtttttttgtttgtttgtttgtttgtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggccaattttttgagtcttttaaagtaaaaatatgtcttgtaagctggtaactatggtacatttccttttattaatgtggtgctgacggtcatataggttcttttgagtttggcatgcatatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgagggaccgtgtcttattcatttccatgtccctagtccatagcccagtgctggacctatggtagtactaaataaatatttgttgaatgcaatagtaaatagcatttcagggagcaagaactagattaacaaaggtggtaaaaggtttggagaaaaaaataatagtttaatttggctagagtatgagggagagtagtaggagacaagatggaaaggtctcttgggcaaggttttgaaggaagttggaagtcagaagtacacaatgtgcatatcgtggcaggcagtggggagccaatgaaggcttttgagcaggagagtaatgtgttgaaaaataaatataggttaaacctatcagagcccctctgacacatacacttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggtaagtggtcatcaatctccaaatccaggttccaggaggttcatgactcccctcccataccccagcctaggctctgttcactcagggaaggaggggagactgtactccccacagaagcccttccagaggtcccataccaatatccccatccccactctcggaggtagaaagggacagatgtggagagaaaataaaaagggtgcaaaaggagagaggtgagctggatgagatgggagagaagggggaggctggagaagagaaagggatgagaactgcagatgagagaaaaaatgtgcagacagaggaaaaaaataggtggagaaggagagtcagagagtttgaggggaagagaaaaggaaagcttgggaggtgaagtgggtaccagagacaagcaagaagagctggtagaagtcatctcatcttaggctacaatgaggaattgagacctaggaagaagggacacagcaggtagagaaacgtggcttcttgactcccaagccaggaatttggggaaaggggttggagaccatacaaggcagagggatgagtggggagaagaaagaagggagaaaggaaagatggtgtactcactcatttgggactcaggactgaagtgcccactcactttttttttttttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacaggggatgaccagagtcattggcgctatggaggtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtggtgagggggtctccccgccgagacttggggatggggcggggcgcagggaagggaaccgtcgcgcagtgcctgcccgggggttgggctggccctaccgggcggggccggctcacttgcctctccctacgcagtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgaggtgagcgcggactggccgagaaggggcaaaggagcggggcggacgggggccagagacgtggccctctcctaccctcgtgtccttttcagatccacgtggttcacctcagcaccgcctttgccagagttgacgaggccttggggcgcccgggaggcctggccgtgttggccgcctttctggaggtaccagatcctggacaccccctactccccgctttcccatcccatgctcctcccggactctatcgtggagccagagaccccatcccagcaagctcactcaggcccctggctgacaaactcattcacgcactgtttgttcatttaacacccactgtgaaccaggcaccagcccccaacaaggattctgaagctgtaggtccttgcctctaaggagcccacagccagtgggggaggctgacatgacagacacataggaaggacatagtaaagatggtggtcacagaggaggtgacacttaaagccttcactggtagaaaagaaaaggaggtgttcattgcagaggaaacagaatgtgcaaagactcagaatatggcctatttagggaatggctacatacaccatgattagaggaggcccagtaaagggaagggatggtgagatgcctgctaggttcactcactcacttttatttatttatttatttttttgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagccacactcactgattctttaatgccagccacacagcacaaagttcagagaaatgcctccatcatagcatgtcaatatgttcatactcttaggttcatgatgttcttaacattaggttcataagcaaaataagaaaaaagaataataaataaaagaagtggcatgtcaggacctcacctgaaaagccaaacacagaatcatgaaggtgaatgcagaggtgacaccaacacaaaggtgtatatatggtttcctgtggggagtatgtacggaggcagcagtgagtgagactgcaaacgtcagaagggcacgggtcactgagagcctagtatcctagtaaagtgggctctctccctctctctccagcttgtcattgaaaaccagtccaccaagcttgttggttcgcacagcaagagtacatagagtttgaaataatacataggattttaagagggagacactgtctctaaaaaaaaaaacaacagcaacaacaaaaagcaacaaccattacaattttatgttccctcagcattctcagagctgaggaatgggagaggactatgggaacccccttcatgttccggccttcagccatggccctggatacatgcactcatctgtcttacaatgtcattcccccaggagggcccggaagaaaacagtgcctatgagcagttgctgtctcgcttggaagaaatcgctgaggaaggtcagtttgttggtctggccactaatctctgtggcctagttcataaagaatcaccctttggagcttcaggtctgaggctggagatgggctccctccagtgcaggagggattgaagcatgagccagcgctcatcttgataataaccatgaagctgacagacacagttacccgcaaacggctgcctacagattgaaaaccaagcaaaaaccgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaaaaaaaaaaaaagaaaaccaagcaaaaaccaaaatgagacaaaaaaaacaagaccaaaaaatggtgtttggaaattgtcaaggtcaagtctggagagctaaactttttctgagaactgtttatctttaataagcatcaaatattttaactttgtaaatacttttgttggaaatcgttctcttcttagtcactcttgggtcattttaaatctcacttactctactagaccttttaggtttctgctagactaggtagaactctgcctttgcatttcttgtgtctgttttgtatagttatcaatattcatatttatttacaagttattcagatcattttttcttttcttttttttttttttttttttttttacatctttagtagagacagggtttcaccatattggccaggctgctctcaaactcctgaccttgtgatccaccagcctcggcctcccaaagtgctgggattcattttttctttttaatttgctctgggcttaaacttgtggcccagcactttatgatggtacacagagttaagagtgtagactcagacggtctttcttctttccttctcttccttcctcccttccctcccaccttcccttctctccttcctttctttcttcctctcttgcttcctcaggcctcttccagttgctccaaagccctgtacttttttttgagttaacgtcttatgggaagggcctgcacttagtgaagaagtggtctcagagttgagttaccttggcttctgggaggtgaaactgtatccctataccctgaagctttaagggggtgcaatgtagatgagaccccaacatagatcctcttcacaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcaggtgggcctggggtgtgtgtggacacagtgggtgcgggggaaagaggatgtaagatgagatgagaaacaggagaagaaagaaatcaaggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaaatcaagaggctggatggggaatacaggagctggagggtggagccctgaggtgctggttgtgagctggcctgggacccttgtttcctgtcatgccatgaacccacccacactgtccactgacctccctagctccacaccctctctgacaccctgtggggacctggtgactctcggctacagctgaacttccgagcgacgcagcctttgaatgggcgagtgattgaggcctccttccctgctggagtggacagcagtcctcgggctgctgagccaggtacagctttgtctggtttccccccagccagtagtcccttatcctcccatgtgtgtgccagtgtctgtcattggtggtcacagcccgcctctcacatctcctttttctctccagtccagctgaattcctgcctggctgctggtgagtctgcccctcctcttggtcctgatgccaggagactcctcagcaccattcagccccagggctgctcaggaccgcctctgctccctctccttttctgcagaacagaccccaaccccaatattagagaggcagatcatggtggggattcccccattgtccccagaggctaattgattagaatgaagcttgagaaatctcccagcatccctctcgcaaaagaatccccccccctttttttaaagatagggtctcactctgtttgccccaggctggggtgttgtggcacgatcatagctcactgcagcctcgaactcctaggctcaggcaatcctttcaccttagcttctcaaagcactgggactgtaggcatgagccactgtgcctggccccaaacggcccttttacttggcttttaggaagcaaaaacggtgcttatcttaccccttctcgtgtatccaccctcatcccttggctggcctcttctggagactgaggcactatggggctgcctgagaactcggggcaggggtggtggagtgcactgaggcaggtgttgaggaactctgcagacccctcttccttcccaaagcagccctctctgctctccatcgcaggtgacatcctagccctggtttttggcctcctttttgctgtcaccagcgtcgcgttccttgtgcagatgagaaggcagcacaggtattacactgaccctttcttcaggcacaagcttcccccacccttgtggagtcacttcatgcaaagcgcatgcaaatgagctgctcctgggccagttttctgattagcctttcctgttgtgtacacacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaatatgtgttagtcacctttgttccccaaatcagaaggaggtatttgaatttcctattactgttattagcaccaatttagtggtaatgcatttattctattacagttcggcctccttccacacatcactccaatgtgttgctcc

SEQ ID NO: 5 - DNA, 25 bp, Homo sapiens
tggggttcttgaggatctccaggag

SEQ ID NO: 6 - DNA, 26 bp, Homo sapiens
ctctaacttcagggagccctcttctt

SEQ ID NO: 7 - protein, 59 aa, Homo sapiens
SSGEDDPLGEEDLPSEEDSPREEDPPGEEDLPGEEDLPGEEDLPEVKPKSEEEGSLKLE

SEQ ID NO: 8 - protein, 59 aa, Homo sapiens
SASEEPSPSEVPFPSEEPSPSEEPFPSVRPFPSVVLFPSEEPFPSKEPSPSEEPSASEE

SEQ ID NO: 9 - protein, 37 aa, Homo sapiens
MAPLCPSPWLPLLIPAPAPGLTVQLLLSLLLLMPVHP

SEQ ID NO: 10 - protein, 377 aa, Homo sapiens
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGD

SEQ ID NO: 11 - protein, 20 aa, Homo sapiens
ILALVFGLLFAVTSVAFLVQ

SEQ ID NO: 12 - protein, 25 aa, Homo sapiens
MRRQHRRGTKGGVSYRPAEVAETGA

SEQ ID NO: 13 - protein, 257 aa, Homo sapiens
GDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFP

(19) United States
(12) Patent Application Publication
(10) Pub. No.: US 2008/0038251 A1
(43) Pub. Date: Feb. 14, 2008

(54) MN/CA IX and MAPK inhibition

(76) Inventors: Silvia Pastorekova, Stupava (SK); Jaromir Pastorek, Stupava (SK)

Correspondence Address: Leona L. Lauder;Attorney at Law, Suite 1026, 235 Montgomery Street, San Francisco, CA, 94104-3008, US

(21) Appl. No.: 11/726,065

(22) Filed: Mar. 20, 2007

Related U.S. Application Data
(60) Provisional application No. 60/784,284, filed on Mar. 20, 2006.

(51) Int. Cl. A61K 31/17; A61K 31/44; A61K 31/70; A61K 38/00; A61P 37/00; C12Q 1/68

(52) U.S. Cl. 424/130.1; 435/6; 514/350; 514/44; 514/594

(57) ABSTRACT

The invention is based upon the discovery that the mitogen-activated protein kinase (MAPK) pathway can increase CA9 expression independently of HIF-1, as well as increasing CA9 expression under HIF-1-dependent pathways initiated by hypoxia or high cell density. Disclosed herein are novel therapeutic methods for treating preneoplastic/neoplastic diseases associated with abnormal MN/CA IX expression, using MAPK pathway inhibitors. Preferably, the MAPK pathway inhibitors are raf kinase inhibitors, particularly the raf kinase inhibitor Sorafenib. Further disclosed are methods for patient therapy selection for MAPK pathway inhibitors, preferably in combination with other cancer therapies, based on detection of abnormal MN/CA9 gene expression in preneoplastic/neoplastic tissues.

### FIELD OF THE INVENTION

The present invention is in the general area of medical genetics and in the fields of biochemical engineering, immunochemistry and oncology. More specifically, it relates to the MN gene—a cellular gene considered to be an oncogene, known alternatively as MN/CA9, CA9, or carbonic anhydrase 9, which gene encodes the oncoprotein now known alternatively as the MN protein, the MN/CA IX isoenzyme, MN/CA IX, carbonic anhydrase IX, CA IX, the MN/G250 or the G250 protein.

More specifically, the instant invention is based upon the discovery that inhibition of the mitogen-activated protein kinase (MAPK) pathway, associated with cancer, also inhibits MN gene expression. That discovery has important applications for the therapy of preneoplastic/neoplastic diseases characterized by abnormal MN gene expression, and for making clinical decisions on cancer treatment.

### BACKGROUND OF THE INVENTION

As indicated above, the MN gene and protein are known by a number of alternative names, which names are used herein interchangeably. The MN protein was found to bind zinc and have carbonic anhydrase (CA) activity and is now considered to be the ninth carbonic anhydrase isoenzyme—MN/CA IX or CA IX [Opavsky et al., Genomics, 33: 480-487 (1996)]. According to the carbonic anhydrase nomenclature, human CA isoenzymes are written in capital roman letters and numbers, whereas their genes are written in italic letters and arabic numbers. Alternatively, “MN” is used herein to refer either to carbonic anhydrase isoenzyme IX (CA IX) proteins/polypeptides, or carbonic anhydrase isoenzyme 9 (CA9) gene, nucleic acids, cDNA, mRNA etc. as indicated by the context.

The MN protein has also been identified with the G250 antigen. Uemura et al. [J. Urol. 157 (4 Suppl.): 377 (Abstract 1475; 1997)] states: “Sequence analysis and database searching revealed that G250 antigen is identical to MN, a human tumor-associated antigen identified in cervical carcinoma (Pastorek et al., 1994).”

Zavada et al., International Publication No. WO 93/18152 (published Sep. 16, 1993) and U.S. Pat. No. 5,387,676 (issued Feb. 7, 1995) describe the discovery of the MN gene and protein. The MN gene was found to be present in the chromosomal DNA of all vertebrates tested, and its expression to be strongly correlated with tumorigenicity. In general, oncogenesis may be signified by the abnormal expression of MN/CA IX protein. For example, oncogenesis may be signified: (1) when MN/CA IX protein is present in a tissue which normally does not express MN/CA IX protein to any significant degree; (2) when MN/CA IX protein is absent from a tissue that normally expresses it; (3) when CA9 gene expression is at a significantly increased level, or at a significantly reduced level from that normally expressed in a tissue; or (4) when MN/CA IX protein is expressed in an abnormal location within a cell. WO 93/18152 further discloses, among other MN-related inventions, MN/CA IX-specific monoclonal antibodies (MAbs), including the M75 MAb and the VU-M75 hybridoma that secretes the M75 MAb. The M75 MAb specifically binds to immunodominant epitopes on the proteoglycan (PG) domain of the MN/CA IX proteins.

Zavada et al., International Publication No. WO 95/34650 (published Dec. 21, 1995) provides in FIG. 1 the nucleotide sequences for a full-length MN cDNA [also provided herein in FIG. 1 (SEQ ID NO: 1)] clone isolated as described therein, and the amino acid sequence [also provided herein in FIG. 1 (SEQ ID NO: 2)] encoded by that MN cDNA. WO 95/34650 also provides in FIG. 6 the nucleotide sequence for the MN promoter [also provided herein in FIG. 6 (SEQ ID NO: 3)]. Those MN cDNA, promoter and amino acid sequences are incorporated by reference herein.

Zavada et al., International Publication No. WO 03/100029 (published Dec. 4, 2003) discloses among other MN-related inventions, MN/CA IX-specific MAbs that are directed to non-immunodominant epitopes, including those on the carbonic anhydrase (CA) domain of the MN/CA IX protein. An example of such a MN/CA IX-specific MAb is the V/10 MAb, secreted from the V/10-VU hybridoma.

The MN protein is now considered to be the first tumor-associated carbonic anhydrase isoenzyme that has been described. The carbonic anhydrase family (CA) includes eleven catalytically active zinc metalloenzymes involved in the reversible hydration-dehydration of carbon dioxide: CO₂+H₂O [symbol] HCO₃⁻+H⁺. CAs are widely distributed in different living organisms. The CAs participate in a variety of physiological and biological processes and show remarkable diversity in tissue distribution, subcellular localization, and biological functions [Parkkila and Parkkila, Scand J Gastroenterol., 31: 305-317 (1996); Potter and Harris, Br J Cancer, 89: 2-7 (2003); Wingo et al., Biochem Biophys Res Commun, 288: 666-669 (2001)]. Carbonic anhydrase IX, CA IX, is one of the most recently identified isoenzymes [Opavsky et al., Genomics, 33: 480-487 (1996); Pastorek et al., Oncogene, 9: 2877-2888 (1994)]. Because of the CA IX overexpression in transformed cell lines and in several human malignancies, it has been recognized as a tumor-associated antigen and linked to the development of human cancers [Zavada et al., Int. J. Cancer, 54: 268-274 (1993); Liao et al., Am. J. Pathol., 145: 598-609 (1994); Saarnio et al., Am J Pathol, 153: 279-285 (1998)].

MN/CA IX is a glycosylated transmembrane CA isoform with a unique N-terminal proteoglycan-like extension. Through transfection studies it has been demonstrated that MN/CA IX can induce the transformation of 3T3 cells [Opavsky et al., Genomics, 33: 480-487 (1996); Pastorek et al., Oncogene, 9: 2877-2888 (1994)].

The MN protein was first identified in HeLa cells, derived from a human carcinoma of cervix uteri. Many studies, using the MN-specific monoclonal antibody (MAb) M75, have confirmed the diagnostic/prognostic utility of MN in diagnosing/prognosing precancerous and cancerous cervical lesions [Liao et al., Am. J. Pathol., 145: 598-609 (1994); Liao and Stanbridge, Cancer Epidemiology, Biomarkers & Prevention, 5: 549-557 (1996); Brewer et al., Gynecologic Oncology 63: 337-344 (1996)]. Immunohistochemical studies with the M75 MAb of cervical carcinomas and a PCR-based (RT-PCR) survey of renal cell carcinomas have identified MN expression as closely associated with those cancers and confirm MN's utility as a tumor biomarker [Liao et al., Am. J. Pathol., 145: 598-609 (1994); Liao and Stanbridge, Cancer Epidemiology, Biomarkers & Prevention, 5: 549-557 (1996); McKiernan et al., Cancer Res. 57: 2362-2365 (1997)]. In various cancers (notably uterine cervical, ovarian, endometrial, renal, bladder, breast, colorectal, lung, esophageal, head and neck and prostate cancers, among others), MN/CA IX expression is increased and has been correlated with microvessel density and the levels of hypoxia in some tumors [Koukourakis et al., Clin Cancer Res, 7: 3399-3403 (2001); Giatromanolaki et al., Cancer Res, 61: 7992-7998 (2001)].

In tissues that normally do not express MN protein, MN/CA IX positivity is considered to be diagnostic for preneoplastic/neoplastic diseases, such as, lung, breast and cervical precancers/cancers [Swinson et al., J Clin Oncol, 21: 473-482 (2003); Chia et al., J Clin Oncol, 19: 3660-3668 (2001); Loncaster et al., Cancer Res, 61: 6394-6399 (2001)], among other precancers/cancers. Very few normal tissues have been found to express MN protein to any significant degree. Those MN-expressing normal tissues include the human gastric mucosa and gallbladder epithelium, and some other normal tissues of the alimentary tract. Paradoxically, MN gene expression has been found to be lost or reduced in carcinomas and other preneoplastic/neoplastic diseases in some tissues that normally express MN, e.g., gastric mucosa.

MN Regulation Under Hypoxia and Normoxia

Strong association of MN/CA IX with a broad range of tumors is principally related to its transcriptional regulation by hypoxia and high cell density, which appear to activate the MN/CA9 promoter through two different, but interconnected pathways [Wykoff et al., Cancer Res., 60: 7075-7083 (2000); Lieskovska, et al., Neoplasma, 46: 17-24 (1999); Kaluz et al., Cancer Res., 62: 4469-4477 (2002)]. Those two pathways are activated via stabilization of HIF-1α by hypoxia, and direct stimulation of MN/CA IX protein expression by the phosphotidylinositol-3-kinase (PI3K) pathway, respectively.

Hypoxia is a reduction in the normal level of tissue oxygen tension. It occurs during acute and chronic vascular disease, pulmonary disease and cancer, and produces cell death if prolonged. Pathways that are regulated by hypoxia include angiogenesis, glycolysis, growth-factor signaling, immortalization, genetic instability, tissue invasion and metastasis, apoptosis and pH regulation [Harris, A. L., Nature Reviews, 2: 38-47 (January 2002)].

The central mediator of transcriptional up-regulation of a number of genes during hypoxia is the transcription factor. HIF-1 is composed of two subunits: a constitutively expressed HIF-1β and a rate-limiting HIF-1α, which is regulated by the availability of oxygen. Under hypoxia, HIF-1α skips modification of its conserved proline and asparagine residues by oxygen-sensitive hydroxylases, thus avoiding degradation mediated by pVHL and inactivation mediated by FIH-1 (factor inhibiting HIF-1) [Maxwell et al., Nature, 399: 271-275 (1999); Jaakkola et al., Science, 292: 468-472 (2001); Ivan et al., Science, 292: 464-468, 2001; Jaakkola, et al., Science, 292: 468-472 (2001); Mahon, et al., Genes Dev., 15: 2675-2686 (2001)]. This leads to HIF-1α accumulation, dimerization with HIF-1β, binding to HRE sites in the target genes, interaction with the cofactors and stimulation of the HIF-1 trans-activation capacity.

In the absence of oxygen, HIF-1 binds to HIF-binding sites within hypoxia-response elements (HRES) of oxygen-regulated genes, thereby activating the expression of numerous hypoxia-response genes, such as erythropoietin (EPO), and the proangiogenic growth factor vascular endothelial growth factor (VEGF). In addition, HIF-1α can be up-regulated under normoxic conditions by different extracellular signals and oncogenic changes transmitted via the PI3K and MAPK pathways [Semenza, Biochem. Pharmacol., 64: 993-998 (2002); Bardos and Ashcroft, BioEssays, 26: 262-269 (2004)]. Whereas PI3K activation results in an increased level of HIF-1α protein, MAPK activation improves its trans-activation properties [Laughner, et al., Mol. Cell. Biol., 21: 3995-4004 (2001); Richard et al., J. Biol. Chem., 274: 32631-32637 (1999)].

MN/CA IX was shown to be one of the most strongly hypoxia-inducible proteins, via the HIF-1 protein binding to the hypoxia-responsive element of the MN promoter [Wykoff et al., Cancer Res, 60: 7075-7083 (2000); Svastova et al., Exp Cell Res, 290: 332-345 (2003)]. Like other HIF-1-regulated genes, the transcription of the MN gene is negatively regulated by wild-type von Hippel-Lindau tumor suppressor gene [Ivanov et al., Proc Natl Acad Sci (USA), 95: 12596-12601 (1998)]. Thus, low levels of oxygen lead to stabilization of HIF-1α, which in turn leads to the increased expression of MN [Wykoff et al., Cancer Res, 60: 7075-7083 (2000)]. Areas of high expression of MN in cancers are linked to tumor hypoxia as reported in many cancers, and incubation of tumor cells under hypoxic conditions leads to the induction of MN expression [Wykoff et al., Cancer Res, 60: 7075-7083 (2000); Koukourakis et al., Clin Cancer Res, 7: 3399-3403 (2001); Giatromanolaki et al., Cancer Res, 61: 7992-7998 (2001); Swinson et al., J Clin Oncol, 21: 473-482 (2003); Chia et al., J Clin Oncol, 19: 3660-3668 (2001); Loncaster et al., Cancer Res, 61: 6394-6399 (2001)].

Key elements of the MN/CA9 promoter are the HIF-1 and SP1 binding regions [Kaluz et al., Cancer Res. 63: 917-922 (2003)] [PR1-HRE element]. The MN/CA9 promoter sequence (−3/−10) between the transcription start and PR1 contains a HRE element recognized by a hypoxia inducible factor 1 (HIF-1), which governs transcriptional responses to hypoxia [Wykoff et al., Cancer Res. 60: 7075-7083 (2000)]. The promoter of the CA9 gene contains five regions protected in DNase I footprinting (PR1-PR5, numbered from the transcription start) [Kaluz et al., J. Biol. Chem., 274: 32588-32595 (1999)]. PR1 and PR2 bind SP1/3 and AP1 transcription factors and are critical for the basic activation of CA9 transcription [Kaluz et al., J. Biol. Chem., 274: 32588-32595 (1999); Kaluzova et al., Biochem. J., 359: 669-677 (2001)]. HIF-1 strongly induces transcription of the CA9 gene in hypoxia, but for full induction requires a contribution of the SP1/3 transcription factor binding to PR1 [Wykoff et al., Cancer Res. 60: 7075-7083 (2000); Kaluz, et al., Cancer Res., 63: 917-922 (2003)].

Regulation under normoxia also requires SP1 [Kaluz et al., Cancer Res., 62: 4469-4477 (2002)]. Upregulation of CA9 transcription in increased cell density involves a mild pericellular hypoxia, depends upon cooperation of SP1 with HIF-1 at subhypoxic level and operates via the PI3K pathway [Kaluz et al., Cancer Res., 62: 4469-4477 (2002)]. Hypoxia and cell density act in an additive fashion so that the highest expression of CA9 is achieved under conditions of low oxygen at high density [Kaluz et al., Cancer Res., 62: 4469-4477 (2002)].

MAPK Pathway

As indicated above, the mitogen-activated protein kinase (MAPK) pathway is an important second signal transduction pathway that affects HIF-1α level and activity, and may also affect MN/CA9 expression. Multiple lines of evidence indicate that the MAPK pathway is important in human cancer. This pivotal pathway relays extracellular signals to the nucleus via a cascade of specific phosphorylation events involving Ras, Raf, MEK, and ERK to regulate fundamental cellular processes, including proliferation, differentiation, and cell survival [Kolch, W., Biochem. J, 351: 289-305 (2000); Lu and Xu, IUBMB Life, 58(11): 621-631 (2006)]. Inappropriate Ras activation is associated with nearly a third of all human cancers [Downward, J. Nat Rev Cancer, 3: 11-22 (2003)]. One of the Raf isoforms, B-raf, is mutated in many cancers, including malignant melanoma (27-70%), papillary thyroid cancer (36-53%), ovarian cancer (30%) and colorectal cancer (5-22%), and the mutations are frequently gain-of-function substitutions that result in constitutive activity [Messersmith et al., Clin Adv. Hematol. Oncol., 4(11): 831-836 (2006); Garnett and Marais, Cancer Cell, 6: 313-319 (2004)]. ERK is elevated in nearly 50% of breast cancers and is associated with a poor prognosis [Messersmith et al. (2006)].

Hypoxia activates ERK kinases by inducing their phosphorylation and nuclear translocation [Minet et al., FEBS Lett., 468: 53-58 (2000); Hofer et al., FASEB J., 15: 2715-2717 (2001)]. In turn, ERKs activate HIF-1 by transmitting the phosphorylation signal to HIF-1 and by recruitment and phosphorylation of HIF-1 coactivators. Under normoxic conditions, the MAPK pathway becomes activated by various growth factors, hormones and by high cell density [Lewis et al., Adv. Cancer Res., 74: 49-139 (1998); Sheta et al., Oncogene, 20: 7624-7634 (2001)]. This normoxic activation also activates HIF-1α and stimulates transcription of HIF-1-regulated genes [Richard et al., J Biol Chem, 274: 32631-32637 (1999)]. Depending on the cell type and culture conditions, hypoxia and mitogenic stimulation can work together to enhance the activation of the MAPK pathway and up-regulation of HIF-1 activity.

As described above, transcription of the CA9 gene coding for a tumor-associated carbonic anhydrase IX (CA IX) isoform is regulated by hypoxia via the hypoxia-inducible factor 1 (HIF-1) and by high cell density via the phosphatidylinositol-3-kinase (PI3K) pathway. The instant invention is based on the discovery that in addition to the PI3K pathway, a second major signal transduction pathway can control MN/CA9 gene expression: the mitogen-activated protein kinase (MAPK) pathway, which discovery accounts for previously unexplained MN expression under normoxic conditions. Moreover, activating mutations of various components of both MAPK and PI3K pathways occur in many tumor types [Vogelstein and Kinzler, “Cancer genes and the pathways they control”, Nat. Med., 10: 789-799 (2004)] and may upregulate MN/CA9 gene expression inside and outside of the hypoxic regions and influence intratumoral distribution of MN/CA IX protein. As MN/CA IX is functionally implicated in tumor growth and survival [Svastova et al., FEBS Lett., 577: 439-445 (2004); Robertson et al., Cancer Res., 64: 6160-6165 (2004)], its increased expression may thus have important consequences for tumor biology. The instant invention discloses therapeutic methods targeted to the MAPK pathway which can be used alone, or in combination with other MN-targeted therapies, to treat preneoplastic/neoplastic diseases characterized by abnormal MN expression.

### SUMMARY OF THE INVENTION

The subject invention is based upon the discovery that the MAPK cascade regulates CA9 gene expression independently of HIF-1 levels. As activating mutations of various components of the MAPK pathway occur in many tumor types, they may upregulate CA9 gene expression, and as CA IX is functionally implicated in tumor growth and survival, its increased expression may thus have important consequences for tumor biology. MAPK pathway inhibitors are then a novel therapy for targeting tumors associated with abnormal CA9 expression, usually increased CA9 expression. Said MAPK pathway inhibitors may be targeted to any components of the MAPK pathway, including Ras, Raf, MEK, and ERK. Preferably, said MAPK pathway inhibitors are inhibitors of Raf, more preferably the Raf kinase inhibitor is the multikinase inhibitor Sorafenib. Preferably, said MAPK pathway inhibitors are used in combination with other CA9-targeted therapies, such as CA IX-specific antibodies, CA IX-specific carbonic anhydrase inhibitors, and/or PI3K-targeted therapies, as MAPK-inhibited cells still retain the capacity to induce CA9 transcription in hypoxia and in high cell density. Consequently, the MAPK kinase inhibitors would be expected to be most effective in early stages of preneoplastic/neoplastic diseases associated with abnormal CA9 gene expression, before CA9-expressing cells have become crowded and/or hypoxic.

In one aspect, the instant invention is directed to a method of treating a mammal, preferably a human, for a preneoplastic/neoplastic disease, wherein said disease is characterized by abnormal MN/CA9 gene expression, comprising administering to said mammal a therapeutically effective amount of a composition comprising a MAPK pathway inhibitor. Preferably, said MAPK pathway inhibitor is a raf kinase inhibitor, preferably the bis aryl-urea Sorafenib (BAY 43-9006) or an omega-carboxypyridyl substituted urea. Most preferably, said raf kinase inhibitor is the bis aryl-urea Sorafenib (BAY 43-9006). Said MAPK pathway inhibitor may be administered in an unmodified form, or may be conjugated to an antibody or biologically active antibody fragment which specifically binds MN/CA IX.

Preferably, said therapeutic methods further comprise administering to said mammal radiation and/or a therapeutically effective amount in a physiologically acceptable formulation of one or more of the following compounds selected from the group consisting of: conventional anticancer drugs, chemotherapeutic agents, different inhibitors of cancer-related pathways, bioreductive drugs, gene therapy vectors, CA IX-specific antibodies and CA IX-specific antibody fragments that are biologically active. Preferably, said inhibitors of cancer-related pathways are inhibitors of the PI3K pathway, and/or said gene therapy vectors are targeted to hypoxic tumors.

Said preneoplastic/neoplastic disease characterized by abnormal MN/CA9 gene expression can be that of many different tissues, for example, uterine, cervical, ovarian, endometrial, renal, bladder, breast, colorectal, lung, esophageal, and prostate, among many other tissues. Of particular interest are preneoplatic/neoplastic diseases of the breast, colon, rectum and of the urinary tract, as of the kidney, bladder and urethra. Renal cell carcinoma (RCC), and metastatic breast cancer are just a couple of representative disease characterized by abnormally high levels of MN/CA9 expression. Also, representative are mesodermal tumors, such as neuroblastomas and retinoblastomas; sarcomas, such as osteosarcomas and Ewing's sarcoma; melanomas; and gynecologic preneoplastic/neoplastic diseases, particularly, of the uterine cervix, endometrium and ovaries, more particularly, cervical squamous cell, adrenosquamous, and glandular preneoplastic/neoplastic diseases, including adenocarcinoma, cervical metaplasia, and condylomas.

Exemplary preneoplastic/neoplastic diseases characterized by abnormal MN/CA9 gene expression are selected from the group consisting of mammary, urinary tract, bladder, kidney, ovarian, uterine, cervical, endometrial, squamous cell, adenosquamous cell, vaginal, vulval, prostate, liver, lung, skin, thyroid, pancreatic, testicular, brain, head and neck, mesodermal, sarcomal, stomach, spleen, gastrointestinal, esophageal, and colon preneoplastic/neoplastic diseases. Said disease may be either a normoxic or a hypoxic tumor.

In a second aspect, the invention concerns a method of therapy selection for a human patient with a preneoplastic/neoplastic disease, comprising detecting and quantifying the level of MN/CA9 gene expression in a sample taken from the patient; and deciding to use MAPK pathway-directed therapy to treat the patient based upon abnormal levels of MN/CA9 gene expression in the patient's sample, usually based upon increased levels of MN/CA9 expression above normal MN/CA9 expression levels. Preferably, said MAPK pathway-directed therapy comprises the use of a raf kinase inhibitor; preferably, said raf kinase inhibitor is the bis aryl-urea Sorafenib (BAY 43-9006) or an omega-carboxypyridyl substituted urea. Most preferably, said raf kinase inhibitor is the bis aryl-urea Sorafenib (BAY 43-9006). Said MAPK pathway inhibitor may be administered in an unmodified form, or may be conjugated to an antibody or biologically active antibody fragment which specifically binds MN/CA IX. Said therapeutic methods may further comprise administering to said human one or more additional therapies; preferably, said additional therapies target MN/CA9 expression or MN/CA IX enzymatic activity.

Said preneoplastic/neoplastic sample would preferably be a tissue, cell or body fluid sample. A tissue sample could be, for example, a formalin-fixed, paraffin-embedded tissue sample or a frozen tissue sample, among other tissue samples. A body fluid sample could be, for example, a blood, serum, plasma or urine sample, among other body fluid samples.

Preferably, said detecting and quantifying step comprises immunologically detecting and quantifying the level of MN/CA IX protein in said sample, and may comprise the use of an assay selected from the group consisting of Western blots, enzyme-linked immunosorbent assays, radioimmunoassays, competition immunoassays, dual antibody sandwich assays, immunohistochemical staining assays, agglutination assays, and fluorescent immunoassays. Preferably, said immunologically detecting and quantifying comprises the use of the monoclonal antibody secreted by the hybridoma VU-M75 which has Accession No. ATCC HB 11128.

Aspects of the instant invention disclosed herein are described in more detail below.

### Abbreviations

The following abbreviations are used herein:

aa—amino acid

ATCC—American Type Culture Collection

bp—base pairs

CA—carbonic anhydrase

Ci—curie

cm—centimeter

CS—cumulative survival

C-terminus—carboxyl-terminus

° C.—degrees centigrade

DFO—deferoxamine mesylate

DMOG—dimethyloxalylglycine

DMSO—dimethyl sulfoxide

ds—double-stranded

EDTA—ethylenediaminetetraacetate

ELISA—enzyme-linked immunosorbent assay

EPO—erythropoietin

ERK—extracellular signal-regulated kinase

FCS—fetal calf serum

FIH-1—factor inhibiting HIF-1

HIF—hypoxia-inducible factor

HRE—hypoxia response element

HRP—horseradish peroxidase

IC—intracellular

kb—kilobase

kbp—kilobase pairs

kd or kDa—kilodaltons

M—molar

MAb—monoclonal antibody

MAPK—mitogen-activated protein kinase

MEK—mitogen/extracellular-signal-regulated kinase kinase), also known as map kinase kinase (MKK)

min.—minute(s)

mg—milligram

ml—milliliter

mM—millimolar

MMA—mithramycin A

mmol—millimole

ng—nanogram

nm—nanometer

nM—nanomolar

nt—nucleotide

N-terminus—amino terminus

ORF—open reading frame

PBS—phosphate buffered saline

PCR—polymerase chain reaction

PG—proteoglycan

PI3K—phosphotidylinositol-3-kinase

pl—isoelectric point

RIPA—radioimmunoprecipitation assay

RT-PCR—reverse transcription polymerase chain reaction

SD—standard deviation

SDS—sodium dodecyl sulfate

SDS-PAGE—sodium dodecyl sulfate-polyacrylamide gel electrophoresis

TM—transmembrane

Tris—tris(hydroxymethyl)aminomethane

μCi—microcurie

μg—microgram

μl—microliter

μM—micromolar

VEGF—vascular endothelial growth factor

VHL—von Hippel-Lindau protein

### Cell Lines

CGL1—non-tumorigenic HeLa x normal fibroblast hybrid cells (HeLa D98/AH.2 derivative; do not express CA9 in a sparse culture under normoixa, but CA9 induced under hypoxia);
CGL3—tumorigenic HeLa x normal fibroblast hybrid cells (HeLa D98/AH.2 derivative; express CA9, but level increased by both high density and hypoxia);
HEK293—human embryonic kidney cells (do not express endogenous CA IX protein);
HeLa—aneuploid, epithelial-like cell line isolated from a human cervical adenocarcinoma [Gey et al., Cancer Res., 12: 264 (1952); Jones et al., Obstet. Gynecol., 38: 945-949 (1971)] obtained from Professor B. Korych, [Institute of Medical Microbiology and Immunology, Charles University; Prague, Czech Republic]; and
Ka13/Ka1.13—derivative of CHO-K1 Chinese hamster cells mutant cell functionally defective for the HIF-1α subunit, provided by Dr. Patrick Maxwell [Imperial College of Science, Technology and Medicine, London, UK]; cell line described in Wood et al., J. Biol. Chem., 273: 8360-8368, 1998.

### Nucleotide and Amino Acid Sequence Symbols

The following symbols are used to represent nucleotides herein:

| Base | |
|---|---|
| Symbol | Meaning |
| A | adenine |
| C | cytosine |
| G | guanine |
| T | thymine |
| U | uracil |
| I | inosine |
| M | A or C |
| R | A or G |
| W | A or T/U |
| S | C or G |
| Y | C or T/U |
| K | G or T/U |
| V | A or C or G |
| H | A or C or T/U |
| D | A or G or T/U |
| B | C or G or T/U |
| N/X | A or C or G or T/U |

There are twenty main amino acids, each of which is specified by a different arrangement of three adjacent nucleotides (triplet code or codon), and which are linked together in a specific order to form a characteristic protein. A three-letter or one-letter convention may be used herein to identify said amino acids as follows:

| | 3 Ltr. | 1 Ltr. |
|---|---|---|
| Amino acid name | Abbrev. | Abbrev. |
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Unknown or other | | X |

### BRIEF DESCRIPTION OF THE FIGURES

FIG. 1A-C provides the nucleotide sequence for a MN cDNA [SEQ ID NO: 1] clone isolated. FIGS. 1A-C also sets forth the predicted amino acid sequence [SEQ ID NO: 2] encoded by the cDNA.

FIG. 2A-F provides a 10,898 bp complete genomic sequence of MN [SEQ ID NO: 4]. The base count is as follows: 2654 A; 2739 C; 2645 G; and 2859 T. The 11 exons are in general shown in capital letters, but exon 1 is considered to begin at position 3507 as determined by RNase protection assay.

FIG. 3 is a restriction map of the full-length MN cDNA. The open reading frame is shown as an open box. The thick lines below the restriction map illustrate the sizes and positions of two overlapping cDNA clones. The horizontal arrows indicate the positions of primers R1 [SEQ ID NO: 5] and R2 [SEQ ID NO: 6] used for the 5′ end RACE. Relevant restriction sites are BamHI (B), EcoRV (V), EcoRI (E), PstI (Ps), PvuII (Pv).

FIG. 4 schematically represents the 5′ MN genomic region of a MN genomic clone wherein the numbering corresponds to transcription initiation sites estimated by RACE.

FIG. 5 provides an exon-intron map of the human MN/CA IX gene. The positions and sizes of the exons (numbered, cross-hatched boxes), Alu repeat elements (open boxes) and an LTR-related sequence (first unnumbered stippled box) are adjusted to the indicated scale. The exons corresponding to individual MN/CA IX protein domains are enclosed in dashed frames designated PG (proteoglycan-like domain), CA (carbonic anhydrase domain), TM (transmembrane anchor) and IC (intracytoplasmic tail). Below the map, the alignment of amino acid sequences illustrates the extent of homology between the MN/CA IX protein PG region (aa 53-111) [SEQ ID NO: 7] and the human aggrecan (aa 781-839) [SEQ ID NO: 8].

FIG. 6 is a nucleotide sequence for the proposed promoter of the human MN gene [SEQ ID NO: 3]. The nucleotides are numbered from the transcription initiation site according to RNase protection assay. Potential regulatory elements are overlined. Transcription start sites are indicated by asterisks (RNase protection) and dots (RACE) above the corresponding nucleotides. The sequence of the 1st exon begins under the asterisks. FTP analysis of the MN4 promoter fragment revealed 5 regions (I-V) protected at both the coding and noncoding strands, and two regions (VI and VII) protected at the coding strand but not at the noncoding strand.

FIG. 7 is a diagram of pathways affecting CA9 expression (“Target Gene Expression”). Oxygen and growth factor-regulated signal transduction determine HIF-1α protein expression and transcriptional activity. HIF-1α is induced by hypoxia in all cell types. In contrast, PI3K and MAPK pathways have cell- and stimulus-specific effects (Semenza, 2002, supra).

FIG. 8 shows that inhibition of ERK by U0126 (an ERK inhibitor) results in down-regulation of the PR1-HRE CA9 promoter activity in dense culture of HEK293 cells independently of hypoxia [as shown in Example 1]. The numbers above the columns show CA9 promoter activity in a luciferase-renilla reporter system, expressed as arbitrary luciferase units. The first two columns represent normoxia; the last two columns show that U0126 inhibition of CA9 promoter activity also occurs under DFO-induced hypoxia.

FIG. 9A-B depicts CA9 transcriptional activity by the PR1-HRE promoter, in CGL1 (A) and CGL3 (B) cells treated by the ERK inhibitor U0126 [Example 2]. The numbers above the columns show CA9 promoter activity in a luciferase-renilla reporter system, expressed as arbitrary luciferase units. In CGL1 cells, which do not express CA9 under normoxia, U0126 inhibited CA9 promoter activity under DFO-induced hypoxia (last two columns). In CGL3 cells, which express CA9 under normoxia, the ERK inhibitor suppressed CA9 transcription in CGL3 cells either plated at high density (first two columns) or under DFO-induced hypoxia (last two columns).

FIG. 10 shows transcriptional activity of the PR1-HRE CA9 promoter measured in Ka1.13 cells defective for HIF-1α [Example 3]. Transfection of HIF-1α cDNA led to increased expression of luciferase from PR1-HRE promoter region (last four columns). However, treatment by ERK inhibitor U0126 reduced the CA9 promoter activity in both the presence and absence of HIF-1α. The numbers above the columns show CA9 promoter activity in a luciferase-renilla reporter system, expressed as arbitrary luciferase units; pcDNA3.1 represents a control plasmid, and DFO is a chemical inducer of hypoxia.

FIG. 11 shows PR1-HRE CA9 promoter activity in the presence of dominant-negative ERK mutants evaluated in dense HEK293 cells in normoxia [Example 4]. The cells were transfected with either a dominant negative ERK1 mutant, a dominant negative ERK2 mutant, or control plasmid pcDNA3.1, and CA9 promoter activity determined at 48 hours (A) and 72 hours (B). Only the ERK1 dominant-negative mutant inhibited CA9 promoter activity. The numbers above the columns show CA9 promoter activity in a luciferase-renilla reporter system, expressed as arbitrary luciferase units.

FIG. 12 depicts the effect of MAPK pathway inhibition by U0126 on CA9 promoter activity. Transcriptional activity of PR1-HRE-luc portion of the CA9 promoter was determined in HeLa cells grown in sparse and dense cultures. The cells were co-transfected with PR1-HRE-luc CA9 promoter construct and renilla plasmid, re-plated at different densities, pre-treated with the U0126 MAPK pathway inhibitor and subjected to DFO-induced hypoxia. CA9 promoter activity was measured 48 h after the transfection and calculated as a ratio between the luciferase and renilla-related values. Results are expressed as the percentage of activity obtained in dense normoxic cultures. Bars represent the mean values including standard deviations.

FIG. 13 represents the transcriptional activity of the CA9 promoter in Ka13 cells cotransfected with the empty pcDNA3.1 plasmid (A) and with the plasmid encoding HIF-1α cDNA (B). Transfection with PR1-HRE-luc construct, treatment with U0126 and DFO, cell cultivation and assessment of the CA9 promoter activity was as described in FIG. 12. Results are expressed as the mean percentage of activity obtained in the normoxic cultures transfected with the empty plasmid. Bars represent the mean values including standard deviations.

FIG. 14 shows the influence of dominant-negative mutant of MAPK/ERK1 on the CA9 promoter activity in HEK293 cells. (A) The cells were co-transfected with the PR1-HRE-luc promoter construct and cDNA encoding the dominant-negative mutants of MAP kinases ERK1 (ERK1-DN) and ERK2 (ERK2-DN) and maintained in normoxia under high density. (B) The cells were co-transfected with PR1-HRE-luc and ERK1-DN plasmids and grown in sparse or dense culture under normoxia or hypoxia (1% O₂). Transcriptional activity of the CA9 promoter was determined as described in FIG. 12. Results are expressed as the mean percentage of activity obtained in the cells co-transfected with an empty pcDNA3.1 plasmid that served as a control.

FIG. 15 depicts the expression of CA9 gene in HeLa cells treated with inhibitors of both MAPK (U0126) and PI3K (LY294002) pathways. Effect of inhibitors on the CA9 promoter activity was evaluated in the cells transfected with the PR1-HRE-luc promoter construct, re-plated at high or low density and maintained for 24 h in normoxia or hypoxia (1% O₂). Results are expressed as the mean percentage of activity measured in the non-treated cells and include standard deviations.

FIG. 16 shows the excessive negative effect of SP1 inhibition (by SP1 inhibitor MMA) on the CA9 promoter activity and CA IX protein expression in MAPK- and/or PI3K-inhibited cells. Effect of inhibitors on the CA9 promoter activity was evaluated in HeLa cells transfected with the PR1-HRE-luc promoter construct, re-plated at high/low density and exposed to normoxia/hypoxia (1% O₂). Results are expressed as the mean percentage of the activity measured in the non-treated cells and include standard deviation.

### DETAILED DESCRIPTION

The MN/CA IX protein is functionally implicated in tumorigenesis as part of the regulatory mechanisms that control pH and cell adhesion. MN/CA IX is induced primarily under hypoxia via the HIF-1 pathway; HIF-1 may also be expressed under normoxia by different extracellular signals and oncogenic changes, such as high cell density, transmitted via the PI3K pathway, which can result in increased MN/CA IX expression. Both the HIF-1 and PI3K pathways increase HIF-1 protein levels, which increases can be translated into increased MN/CA IX levels. However, it had been unknown whether the MN/CA9 promoter only responds to increased levels of HIF-1 protein, or could also respond to normoxic changes which only affect HIF-1 activation.

The inventors found, as shown in the Examples below, that the expression of CA9 is subject to regulation by the MAPK pathway independent of HIF-1α levels. The inventors then found another source of increased CA9 expression, that is, a source beyond hypoxia and cell density, a third source via the MAPK pathway.

The invention is also based on the discovery that besides the PI3K pathway, the MAPK cascade regulates MN/CA9 gene expression under both hypoxia and high cell density. Inhibition of the MAPK pathway by a specific inhibitor down-regulated the CA9 promoter activity and CA IX protein expression in both hypoxia and high cell density. As shown in Examples 4 and 7, transcriptional activity of the CA9 promoter was also reduced by expression of a dominant-negative mutant of the ERK1 component of the MAPK pathway. Finally, simultaneous inhibition of both PI3K and MAPK in Example 8 down-regulated the CA9 promoter activity and protein level more strongly than their separate inhibition, indicating their dual control of MN/CA9 gene expression.

MN and Cancer Therapy

Because of MN protein's unique characteristics, it is an attractive candidate target for cancer therapy. In comparison to other tumor-related molecules (e.g. growth factors and their receptors), MN has the unique property of being differentially expressed in preneoplastic/neoplastic and normal tissues. Because of the extremely limited expression of MN protein in normal tissues, chemopreventive agents that target its expression would be expected to have reduced side effects, relative to agents that target proteins more extensively found in normal tissues (e.g., tamoxifen which binds the estrogen receptor, and finasteride which binds the androgen receptor). Furthermore, Phase I and II clinical trials of an MN-specific drug, Rencarex®, have shown that at least one MN-specific agent is well-tolerated, with no serious drug-related side effects, further supporting MN as a possible target for cancer chemoprevention.

MAPK Inhibitors

The invention is based upon the discovery that MAPK pathway inhibitors can inhibit MN/CA9 gene expression, and can therefore be used therapeutically to treat preneoplastic/neoplastic diseases characterized by abnormal MN/CA9 gene expression.

As used herein, “MAPK pathway inhibitors” include any therapies that are targeted to the MAPK pathway, including targeting any of the MAPK components, Ras, Raf, MEK, and ERK, including but not limited to inhibition of their protein expression (e.g., antisense oligonucleotides), prevention of membrane localization essential for MAPK activation, and inhibition of downstream effectors of MAPK (e.g., Raf serine/threonine kinases) [for review of MAPK inhibitors, see Gollob et al., Semin Oncol., 33(4): 392-406 (2006)]. MAPK pathway-directed therapies include but are not limited to multi-kinase inhibitors, tyrosine kinase inhibitors, monoclonal antibodies, as well as biologically active antibody fragments, polyclonal antibodies, and anti-anti-idiotype antibodies and related antibody based therapies, bis-aryl ureas, and omega-carboxypyridyl substituted ureas and the like. Preferred MAPK pathway inhibitors are Raf kinase inhibitors, which are described in more detail below.

Thus far, the most successful clinical drugs targeting the Ras/Raf/MEK/ERK cascade appear to be those that target Raf [Schreck and Rapp, Int. J Cancer, 119: 2261-2271 (2006)], including the multi-kinase inhibitor Sorafenib (BAY 43-9006), and antisense and heat shock protein 90 (HSP90) inhibitors.

An exemplary and preferred MAPK pathway-directed therapy according to the invention is the bis-aryl urea Sorafenib (BAY 43-9006) [Nexavar®; Onyx Pharmaceuticals, Richmond, Calif. (USA), and Bayer Corporation, West Haven, Conn. (USA); Wilhelm and Chien, Curr Pharm Des, 8: 2255-2257 (2002); Wilhelm et al., Cancer Res., 64: 7099-7109 (2004); Strumberg, D, Drugs Today (Barc), 41: 773-84, 2005; Lyons et al., Endocrine-Related Cancer, 8: 219-225 (2001)], a small molecule and novel dual-action inhibitor of both Raf (a protein-serine/threonine kinase) and VEGFR (vascular endothelial growth factor receptor, a receptor tyrosine kinase), and consequently an inhibitor of both tumor cell proliferation and angiogenesis. In addition, Sorafenib has been found to inhibit several other receptor tyrosine kinases involved in tumor progression and neovascularization, including PDGFR-β, Flt-3, and c-KIT. In December 2005 Sorafenib was approved by the FDA for patients with advanced renal cell carcinoma (RCC).

Other exemplary therapies that target the MAPK pathway include MEK inhibitors. PD-0325901 (Pfizer) and ARRY-142886 (AZD-6244, Array and AstraZeneca) are small-molecule inhibitors currently in clinical development [Gollob et al., Semin Oncol., 33(4): 392-406 (2006); Doyle et al., Proc Am Soc Clin Oncol. 24: 3075, 2005 (Abstr.); Lee et al., Cancer, 2: 368 (2004) (Suppl.)] Those two orally available agents are non-ATP competitive allosteric inhibitors of MEK, which unlike the majority of ATP-competitive analogs, show high selectivity for MEK in biochemical assays. PD-0325901 is a second-generation compound derived from CI-1040 (PD184352, Pfizer), an oral MEK inhibitor which began Phase II clinical trials. PD-0325901 has an IC₅₀ value 200-fold lower than CI-1040, and is also more soluble with improved metabolic stability and bioavailability. PD-0325901 and ARRY-142886 have shown potent anti-tumor activity in tumor xenograft models. ARRY-142886 is currently being evaluated in phase 1 trials, while phase I/II clinical trial findings have recently been reported with PD-0325901.

Raf Kinase Inhibitors

As used herein, “raf kinase inhibitors” include any therapies that are targeted to raf expression or activation, including inhibition of Raf protein expression (e.g., antisense oligonucleotides), small molecule inhibitors of Raf serine/threonine kinases, Raf kinase destabilizers (e.g., inhibitors of HSP90 and HDAC), or immune therapies. Small molecule inhibitors of Raf serine/threonine kinases may be, for example, bis-aryl ureas, or omega-carboxypyridyl substituted ureas. Exemplary raf-targeted therapies according to the invention are the small molecule inhibitors Sorafenib and CHIR-265; the antisense inhibitors ISIS 5132 and LErafAON-ETU; the HSP90 inhibitors 17-MG and 17-DMAg; the HDAC inhibitors SAHA and NVPLAQ824 [reviewed in Schreck and Rapp, Int. J Cancer, 119: 2261-2271, 2006]. A preferred raf-directed therapy according to the invention is the bis-aryl urea Sorafenib (BAY 43-9006) [Nexavar®; Onyx Pharmaceuticals, Richmond, Calif. (USA), and Bayer Corporation, West Haven, Conn. (USA); Lyons et al., Endocrine-Related Cancer, 8: 219-225 (2001) and Wilhelm et al. (2004)], a small molecule which inhibits the enzyme Raf kinase. Other preferred raf-directed therapies according to the invention are omega-carboxypyridyl substituted ureas, which are derivatives of bis-aryl ureas with improved solubility in water [Khire et al., Bioorg Med Chem Lett., 14(3): 783-786 (2004)].

Use of MAPK Inhibitors with Conventional or MN-Directed Therapies

According to the methods of the invention, the MAPK inhibitors can be combined with MN/CA IX-specific antibodies and a variety of conventional therapeutic drugs, different inhibitors of cancer-related pathways, bioreductive drugs, and/or radiotherapy, wherein different combinations of treatment regimens with the MAPK inhibitors may increase overall treatment efficacy. Preferred therapies to be used in combination with MAPK inhibitors are inhibitors of the PI3K pathway, as well as MN-directed therapies.

PI3K Pathway Inhibitors

Activation of the phosphotidylinositol-3-kinase (PI3K)/Akt cell survival pathway in many cancers makes it an obvious target for cancer therapy. Because this pathway also has an important role in the survival of normal cells, however, it is important to achieve cancer selectivity; the cancer-selective proapoptotic protein Par-4 is a key target for inactivation by PI3K/Akt signaling [Goswami et al., Cancer Res., 66(6): 2889-2892 (2006)]. Several anticancer therapies target, albeit indirectly, the PI3K/Akt pathway and cause inhibition of Akt1 phosphorylation and induction of apoptosis. Examples include herceptin, which inhibits ErbB-2 in breast cancer cells; cyclooxygenase-2 (CAOX-2) inhibitors, which inhibit COX-2 and PD1 in colon and prostate cancer; gefitnib (Iressa), which targets mutant epidermal growth factor receptor in lung cancer cells; and imatinib mesylate (Gleevec, STI-571), which targets bcr-abl in leukemia.

MN-Directed Therapies

Many MN-directed therapies may be useful according to the methods of the present invention, to be used in combination with MAPK pathway inhibitors to treat preneoplastic/neoplastic diseases associated with abnormal MN expression.

Preferred therapies comprise therapies selected from the group consisting of MN-specific antibodies, MN-preferential carbonic anhydrase inhibitors, MN antisense nucleic acids, MN RNA interference, and MN gene therapy vectors; some of which preferred therapies are described in greater detail below.

Particularly, the MAPK specific inhibitors may be combined with therapy using MN/CA IX-specific antibodies and/or MN/CA IX-specific antibody fragments, preferably humanized MN/CA IX-specific antibodies and/or biologically active fragments thereof, and more preferably fully human MN/CA IX-specific antibodies and/or fully human MN/CA IX-specific biologically active antibody fragments. Said MN/CA IX-specific antibodies and biologically active MN/CA IX-specific antibody fragments, preferably humanized and more preferably fully human, may be conjugated to the MAPK inhibitor, or to a cytotoxic entity, for example, a cytotoxic protein, such as ricin A, among many other cytotoxic entities.

Still further, a MAPK inhibitor of this invention could be administered with a vector targeted for delivery to MN/CA IX-specific expressing cells for gene therapy (for example, with the wild-type von Hippel-Lindau gene), or for effecting the expression of cytotoxic proteins, preferably wherein said vector comprises an MN/CA9 promoter or MN/CA9 promoter fragment comprising the MN/CA9 hypoxia response element (HRE) or a HRE of another gene, and more preferably wherein the MN/CA9 promoter or MN/CA9 promoter fragment comprises more than one HRE, wherein said HRE or HREs is or are either of MN/CA9, and/or of other genes and/or of genetically engineered HRE consensus sequences in a preferred context.

Preneoplastic/Neoplastic Tissues

The novel methods of the present invention inhibit preneoplastic/neoplastic cell growth by preventing MN gene expression using MAPK pathway inhibitors, alone or in combination with MN-directed therapies. Those methods are expected to be effective for any preneoplastic/neoplastic disease characterized by abnormal MN/CA9 gene expression. Exemplary preneoplastic/neoplastic diseases include at the least preneoplastic/neoplastic diseases selected from the group consisting of mammary, urinary tract, bladder, kidney, ovarian, uterine, cervical, endometrial, squamous cell, adenosquamous cell, vaginal, vulval, prostate, liver, lung, skin, thyroid, pancreatic, testicular, brain, head and neck, mesodermal, sarcomal, stomach, spleen, gastrointestinal, esophageal, colorectal and colon preneoplastic/neoplastic diseases.

As used herein, “cancerous” and “neoplastic” have equivalent meanings, and “precancerous” and “preneoplastic” have equivalent meanings.

Assays to Screen for MN/CA9 Gene Expression in Tissues

The methods may comprise screening for MN/CA9 gene expression product, if any, present in a sample taken from a patient diagnosed with preneoplastic/neoplastic disease; the MN/CA9 gene expression product can be MN protein, MN polypeptide, mRNA encoding a MN protein or polypeptide, a cDNA corresponding to an mRNA encoding a MN protein or polypeptide, or the like. If the MN/CA9 gene expression product is present at abnormal levels in said sample, the patient may be a suitable candidate for the therapeutic methods of the invention. In most cases, the abnormal levels would be increased MN/CA9 expression levels in tissues that do not normally express MN.

In a preferred embodiment of the invention, the MN gene expression product is MN antigen, and the presence or absence of MN antigen is screened in preneoplastic/neoplastic mammalian samples, preferably human samples. Such preneoplastic/neoplastic samples can be tissue specimens, tissue extracts, cells, cell lysates and cell extracts, among other samples. Preferred tissue samples are formalin-fixed, paraffin-embedded tissue samples or frozen tissue samples.

It can be appreciated by those of skill in the art that various other preneoplastic/neoplastic samples can be used to screen for the MN gene expression products. For example, in the case of a patient afflicted with a neoplastic disease, wherein the disease is a tumor, the sample may be taken from the tumor or from a metastatic lesion derived from the tumor.

It can further be appreciated that alternate methods, in addition to those disclosed herein, can be used to quantify the MN gene expression products.

In preferred embodiments, the gene expression product is MN antigen which is detected by immunohistochemical staining (e.g., using tissue arrays or the like). Preferred tissue specimens to assay by immunohistochemical staining, for example, include cell smears, histological sections from biopsied tissues or organs, and imprint preparations among other tissue samples. Such tissue specimens can be variously maintained, for example, they can be fresh, frozen, or formalin-, alcohol- or acetone- or otherwise fixed and/or paraffin-embedded and deparaffinized. Biopsied tissue samples can be, for example, those samples removed by aspiration, bite, brush, cone, chorionic villus, endoscopic, excisional, incisional, needle, percutaneous punch, and surface biopsies, among other biopsy techniques.

Many formats can be adapted for use with the methods of the present invention. The detection and quantitation of MN protein or MN polypeptide can be performed, for example, by Western blots, enzyme-linked immunosorbent assays, radioimmunoassays, competition immunoassays, dual antibody sandwich assays, immunohistochemical staining assays, agglutination assays, fluorescent immunoassays, immunoelectron and scanning microscopy using immunogold, among other assays commonly known in the art. The detection of MN gene expression products in such assays can be adapted by conventional methods known in the art.

It is also apparent to one skilled in the art of immunoassays that MN proteins or polypeptides can be used to detect and quantitate MN antigen in body tissues and/or cells of patients. In one such embodiment, an immunometric assay may be used in which a labelled antibody made to MN protein is used. In such an assay, the amount of labelled antibody which complexes with the antigen-bound antibody is directly proportional to the amount of MN antigen in the sample.

MN Gene and Protein

The terms “CA IX” and “MN/CA9” are herein considered to be synonyms for MN. Also, the G250 antigen is considered to refer to MN protein/polypeptide [Jiang et al., PNAS (USA) 97: 1749-173 (2000)].

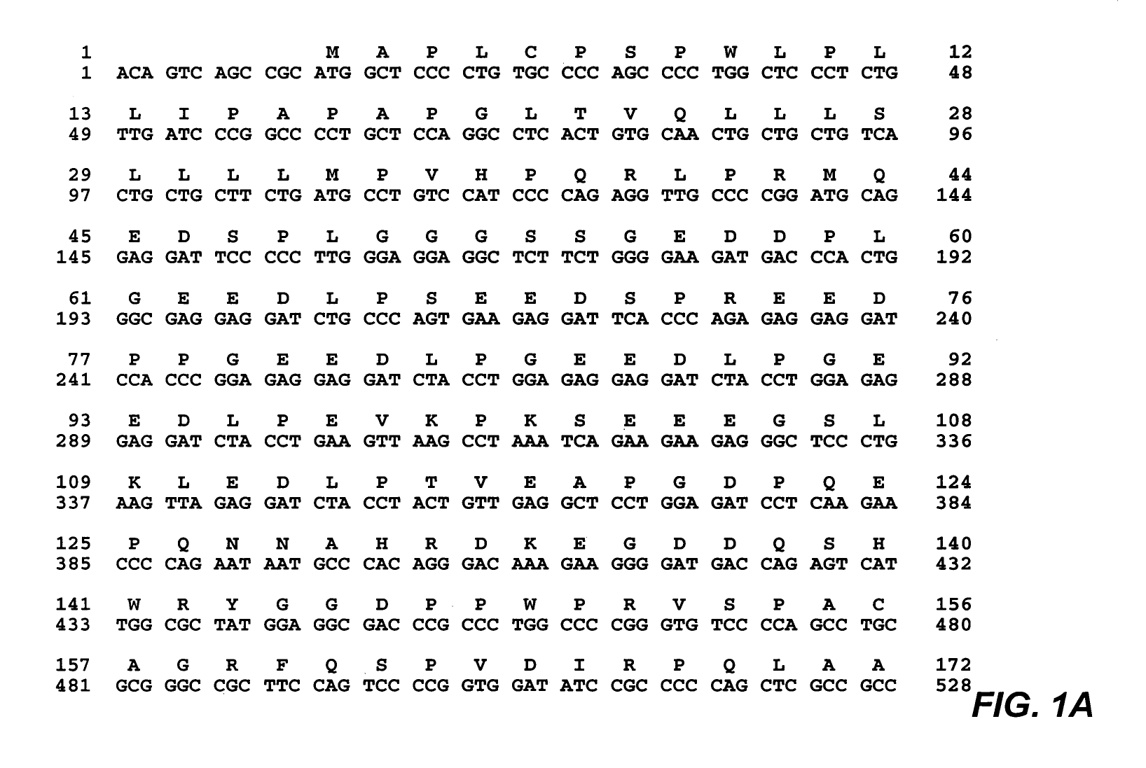

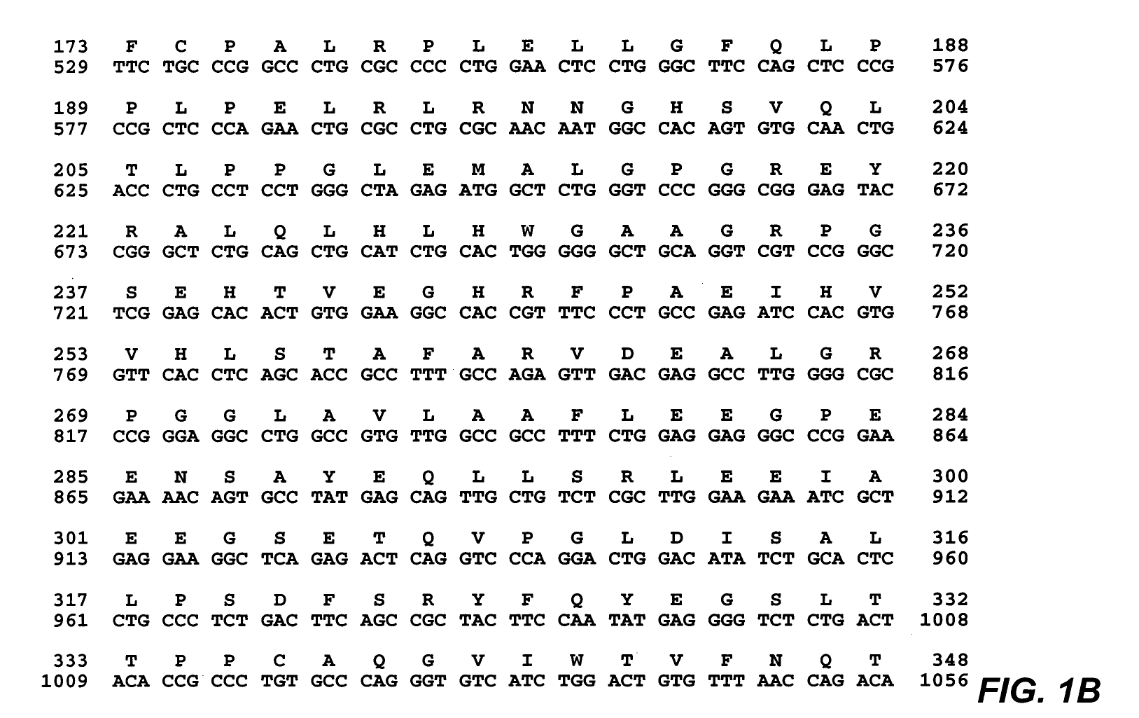

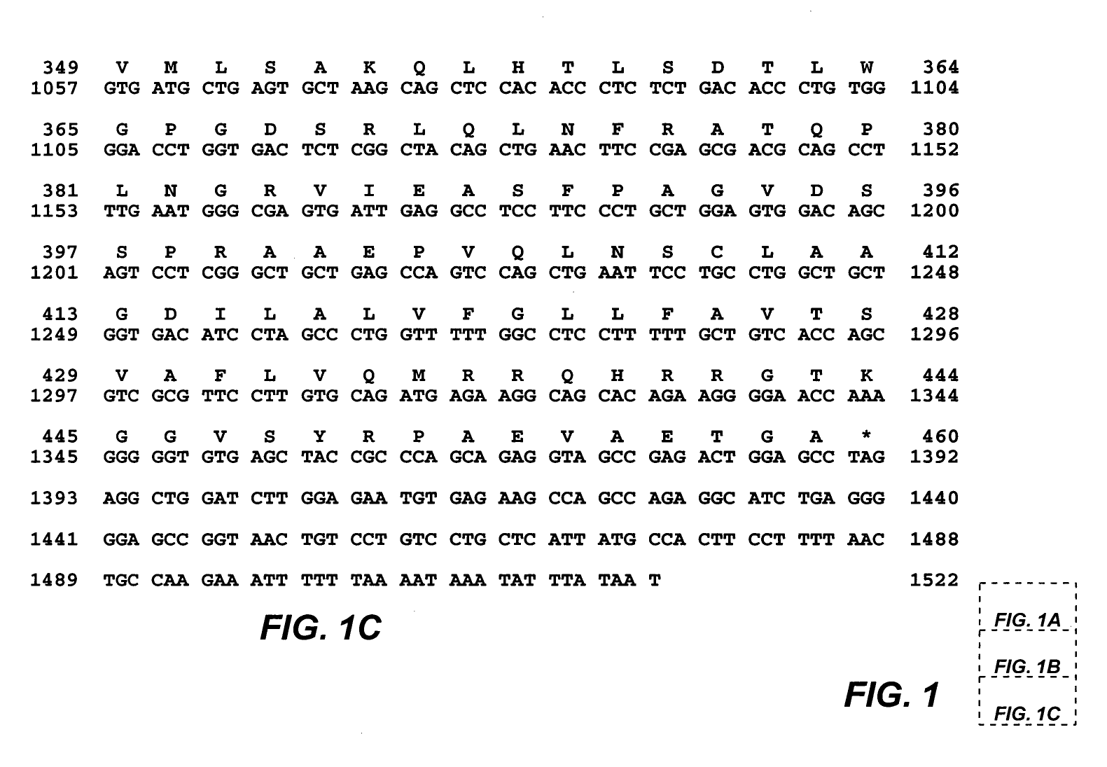

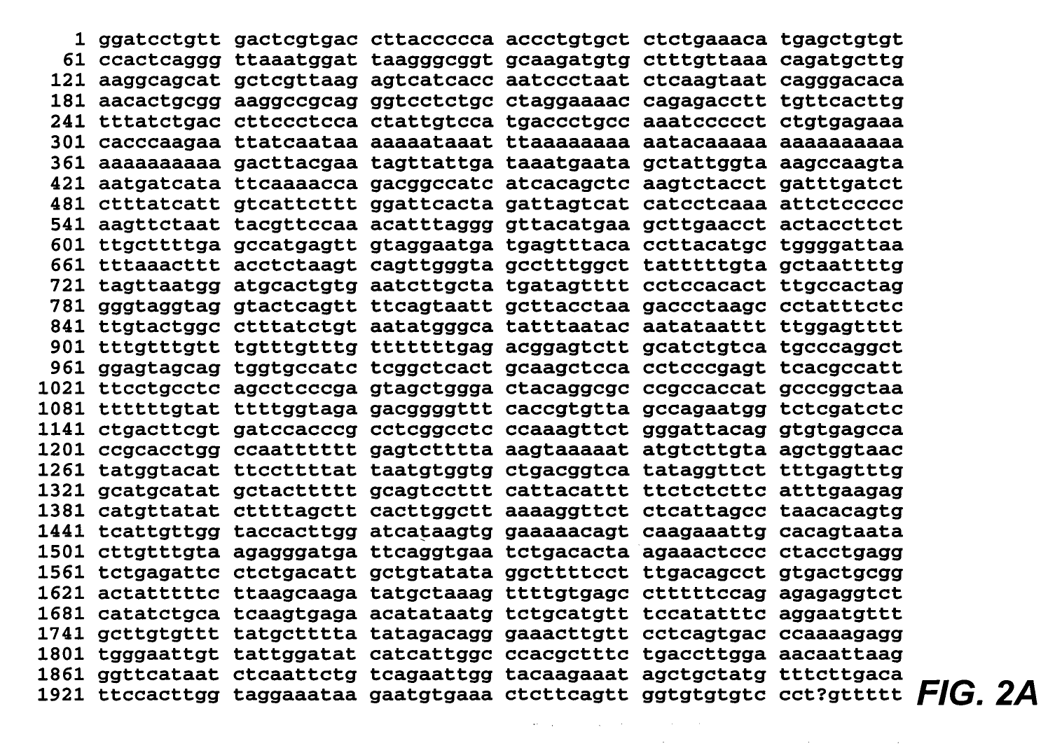

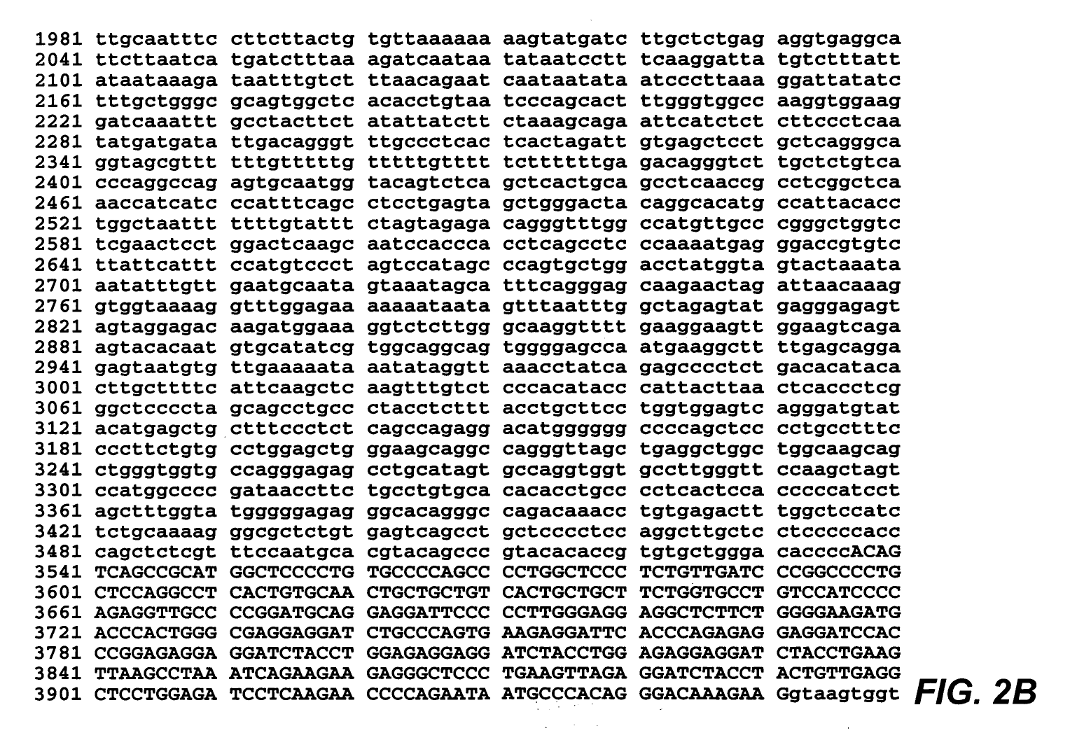

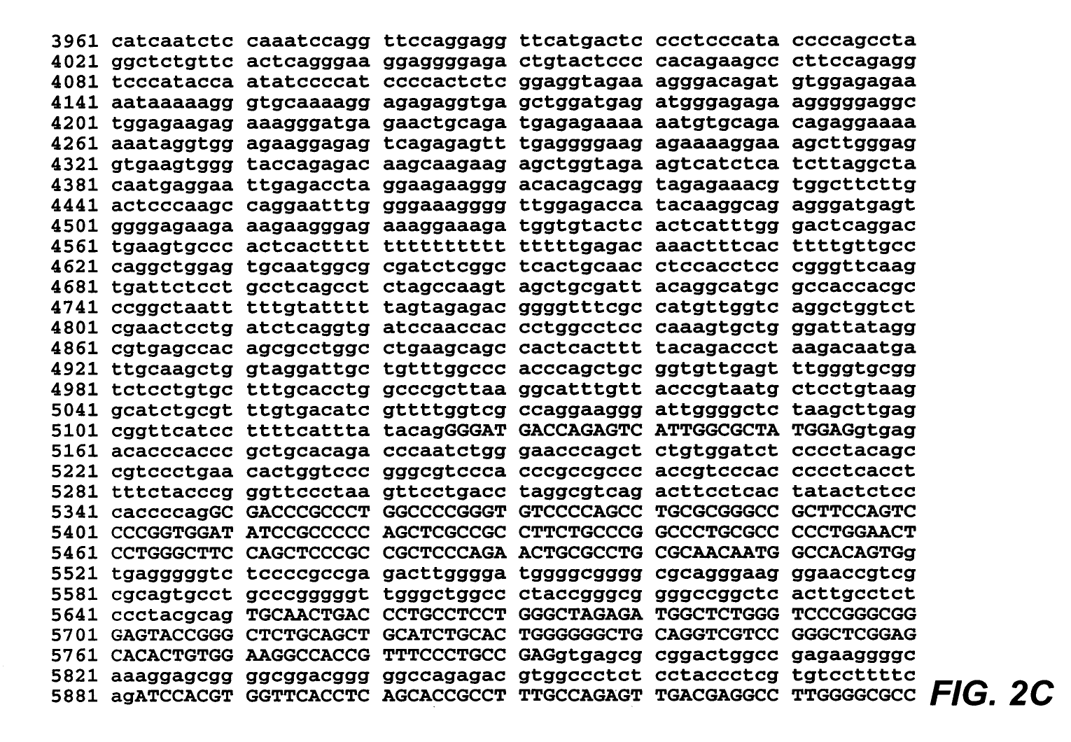

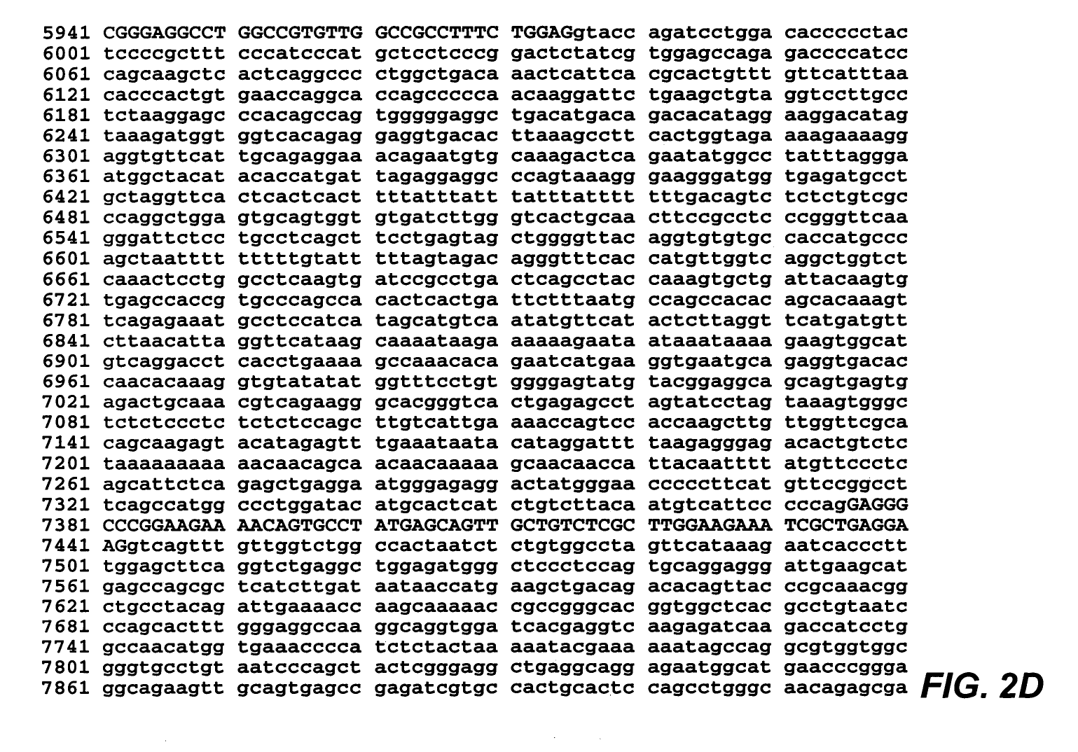

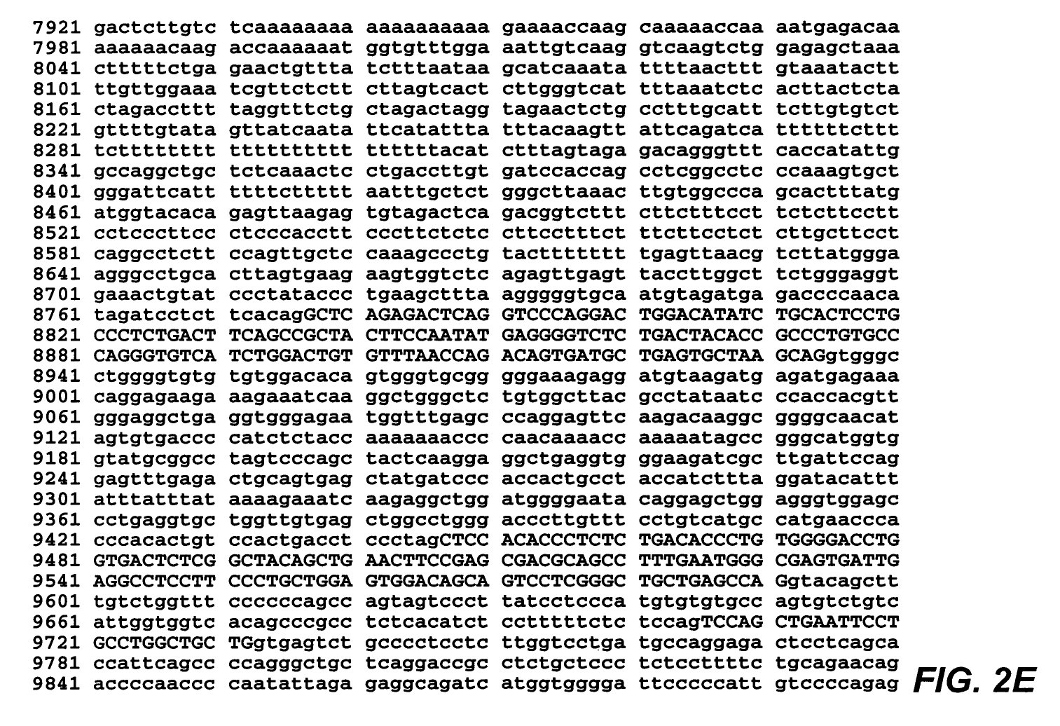

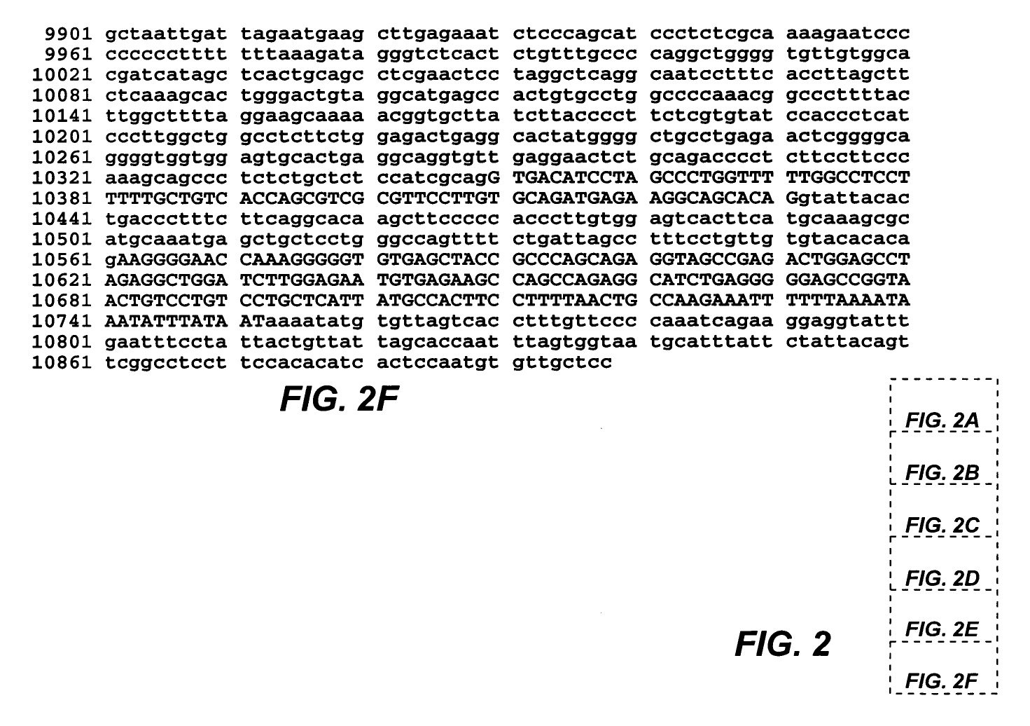

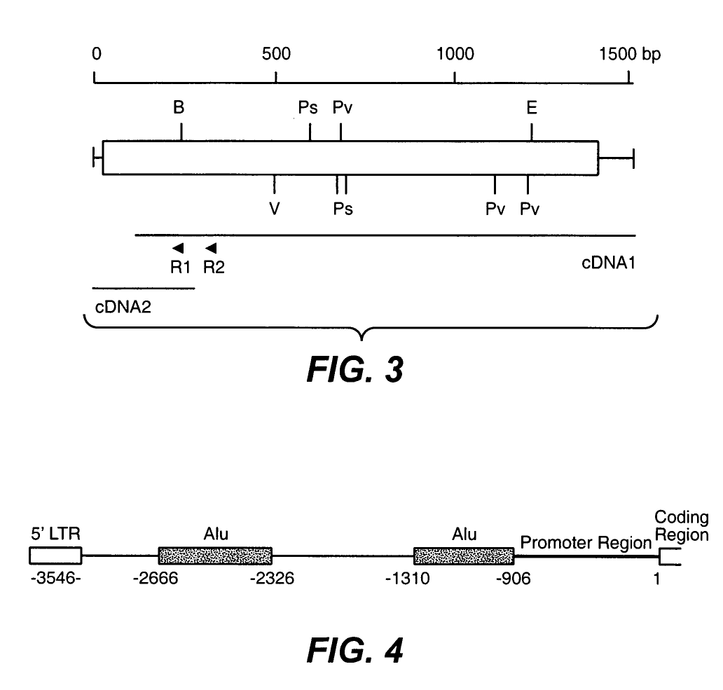

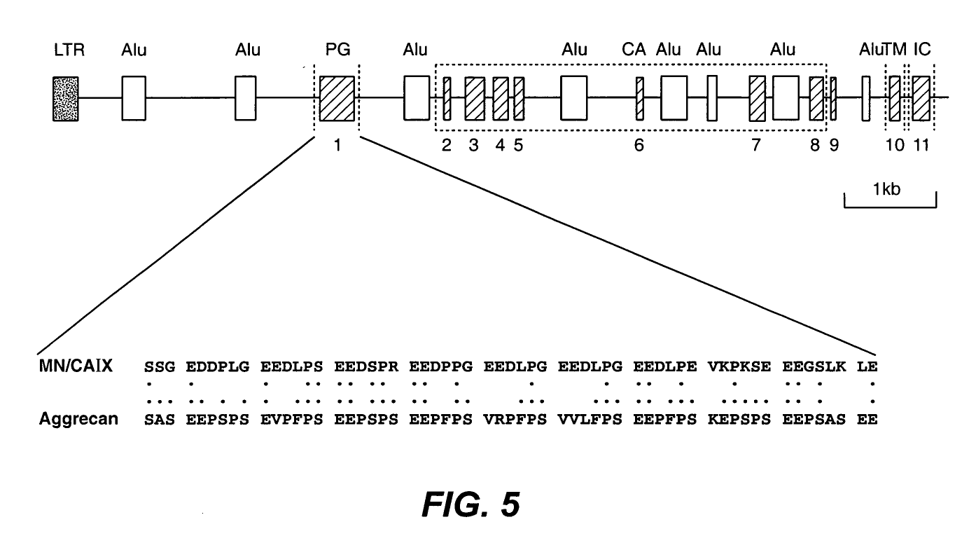

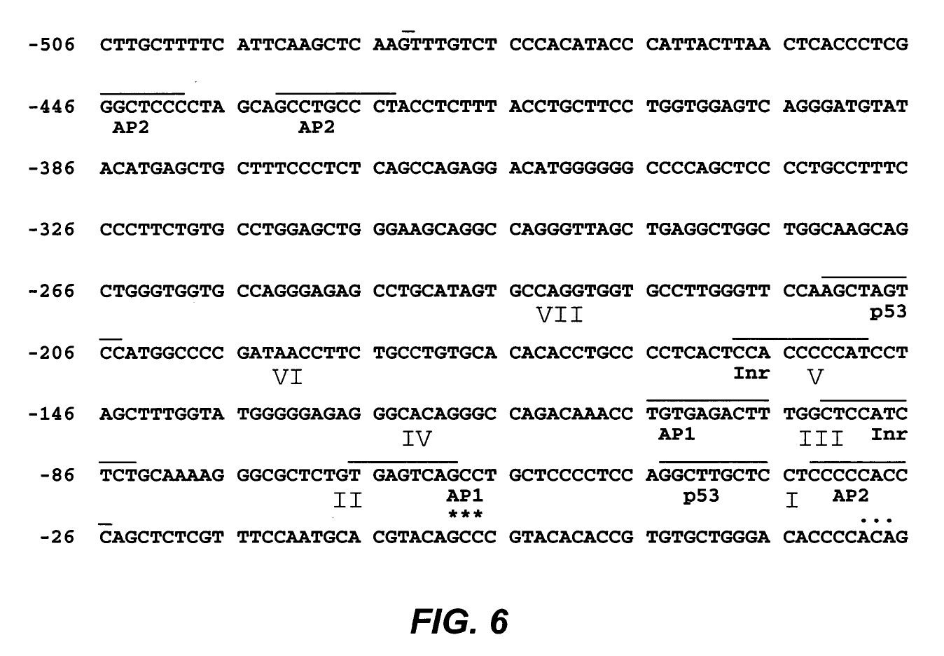

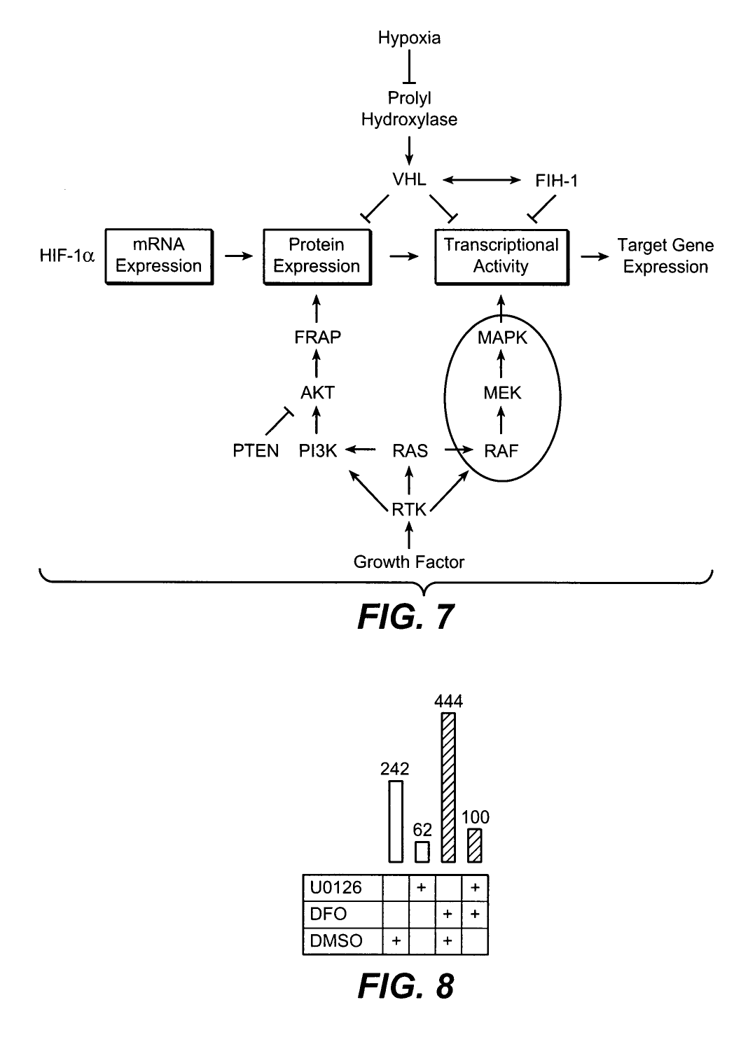

Zavada et al., WO 93/18152 and/or WO 95/34650 disclose the MN cDNA sequence shown herein in FIGS. 1A-1C [SEQ ID NO: 1], the MN amino acid sequence [SEQ ID NO: 2] also shown in FIGS. 1A-1C, and the MN genomic sequence [SEQ ID NO: 4] shown herein in FIGS. 2A-2F. The MN gene is organized into 11 exons and 10 introns.

The ORF of the MN cDNA shown in FIG. 1 has the coding capacity for a 459 amino acid protein with a calculated molecular weight of 49.7 kd. The overall amino acid composition of the MN protein is rather acidic, and predicted to have a pl of 4.3. Analysis of native MN protein from CGL3 cells by two-dimensional electrophoresis followed by immunoblotting has shown that in agreement with computer prediction, the MN is an acidic protein existing in several isoelectric forms with pls ranging from 4.7 to 6.3.

The first thirty seven amino acids of the MN protein shown in FIGS. 1A-1C is the putative MN signal peptide [SEQ ID NO: 9]. The MN protein has an extracellulardomain [amino acids (aa) 38-414 of FIGS. 1A-1C [SEQ ID NO: 10], a transmembrane domain [aa 415-434; SEQ ID NO: 11] and an intracellular domain [aa 435-459; SEQ ID NO: 12]. The extracellular domain contains the proteoglycan-like domain [aa 53-111: SEQ ID NO: 7] and the carbonic anhydrase (CA) domain [aa 135-391; SEQ ID NO: 13].

The CA domain is essential for induction of anchorage independence, whereas the TM anchor and IC tail are dispensable for that biological effect. The MN protein is also capable of causing plasma membrane ruffling in the transfected cells and appears to participate in their attachment to the solid support. The data evince the involvement of MN in the regulation of cell proliferation, adhesion and intercellular communication.

MN Proteins and Polypeptides

The phrase “MN proteins and/or polypeptides” (MN proteins/polypeptides) is herein defined to mean proteins and/or polypeptides encoded by an MN gene or fragments thereof. An exemplary and preferred MN protein according to this invention has the deduced amino acid sequence shown in FIG. 1. Preferred MN proteins/polypeptides are those proteins and/or polypeptides that have substantial homology with the MN protein shown in FIG. 1. For example, such substantially homologous MN proteins/polypeptides are those that are reactive with the MN-specific antibodies, preferably the Mab M75 or its equivalent. The VU-M75 hybridoma that secretes the M75 Mab was deposited at the ATCC under HB 11128 on Sep. 17, 1992.

A “polypeptide” or “peptide” is a chain of amino acids covalently bound by peptide linkages and is herein considered to be composed of 50 or less amino acids. A “protein” is herein defined to be a polypeptide composed of more than 50 amino acids. The term polypeptide encompasses the terms peptide and oligopeptide.

It can be appreciated that a protein or polypeptide produced by a neoplastic cell in vivo could be altered in sequence from that produced by a tumor cell in cell culture or by a transformed cell. Thus, MN proteins and/or polypeptides which have varying amino acid sequences including without limitation, amino acid substitutions, extensions, deletions, truncations and combinations thereof, fall within the scope of this invention. It can also be appreciated that a protein extant within body fluids is subject to degradative processes, such as, proteolytic processes; thus, MN proteins that are significantly truncated and MN polypeptides may be found in body fluids, such as, sera. The phrase “MN antigen” is used herein to encompass MN proteins and/or polypeptides.

It will further be appreciated that the amino acid sequence of MN proteins and polypeptides can be modified by genetic techniques. One or more amino acids can be deleted or substituted. Such amino acid changes may not cause any measurable change in the biological activity of the protein or polypeptide and result in proteins or polypeptides which are within the scope of this invention, as well as, MN muteins.

Antibodies

The term “antibodies” is defined herein to include not only whole antibodies but also biologically active fragments of antibodies, preferably fragments containing the antigen binding regions. Further included in the definition of antibodies are bispecific antibodies that are specific for MN protein and to another tissue-specific antigen.

Antibodies useful according to the methods of the invention may be prepared by conventional methodology and/or by genetic engineering. Antibody fragments may be genetically engineered, preferably from the variable regions of the light and/or heavy chains (V_{H} and V_{L}), including the hypervariable regions, and still more preferably from both the V_{H} and V_{L} regions. For example, the term “antibodies” as used herein includes polyclonal and monoclonal antibodies and biologically active fragments thereof including among other possibilities “univalent” antibodies [Glennie et al., Nature, 295: 715 (1982)]; Fab proteins including Fab′ and F(ab)₂ fragments whether covalently or non-covalently aggregated; light or heavy chains alone, preferably variable heavy and light chain regions (V_{H} and V_{L} regions), and more preferably including the hypervariable regions [otherwise known as the complementarity determining regions (CDRs) of the V_{H} and V_{L} regions]; F_{c} proteins; “hybrid” antibodies capable of binding more than one antigen; constant-variable region chimeras; “composite” immunoglobulins with heavy and light chains of different origins; “altered” antibodies with improved specificity and other characteristics as prepared by standard recombinant techniques and also oligonucleotide-directed mutagenesis techniques [Dalbadie-McFarland et al., PNAS (USA, 79: 6409 (1982)].

The antibodies useful according to this invention to identify MN proteins/polypeptides can be labeled in any conventional manner, for example, with enzymes such as horseradish peroxidase (HRP), fluorescent compounds, or with radioactive isotopes such as, ¹²⁵I, among other labels. A preferred label, according to this invention is ¹²⁵I, and a preferred method of labeling the antibodies is by using chloramine-T [Hunter, W. M., “Radioimmunoassay,” In: Handbook of Experimental Immunology, pp. 14.1-14.40 (D. W. Weir ed.; Blackwell, Oxford/London/Edinburgh/Melbourne; 1978)].

Representative monoclonal antibodies useful according to this invention include Mabs M75, MN9, MN12 and MN7 described in earlier Zavada et al. patents and patent applications. [U.S. Pat. No. 6,297,041; U.S. Pat. No. 6,204,370; U.S. Pat. No. 6,093,548; U.S. Pat. No. 6,051,226; U.S. Pat. No. 6,004,535; U.S. Pat. No. 5,989,838; U.S. Pat. No. 5,981,711; U.S. Pat. No. 5,972,353; U.S. Pat. No. 5,955,075; U.S. Pat. No. 5,387,676; US Application Nos: 20050031623, 20030049828, and 20020137910; and International Publication No. WO 03/100029]. Monoclonal antibodies useful according to this invention serve to identify MN proteins/polypeptides in various laboratory prognostic tests, for example, in clinical samples. For example, monoclonal antibody M75 (Mab M75) is produced by mouse lymphocytic hybridoma VU-M75, which was deposited under ATCC designation HB 11128 on Sep. 17, 1992 at the American Tissue Type Culture Collection [ATCC]. The production of hybridoma VU-M75 is described in Zavada et al., International Publication No. WO 93/18152. Mab M75 recognizes both the nonglycosylated GST-MN fusion protein and native MN protein as expressed in CGL3 cells equally well. The M75 Mab recognizes both native and denatured forms of the MN protein [Pastorekova et al., Virology, 187: 620-626 (1992)].

General texts describing additional molecular biological techniques useful herein, including the preparation of antibodies include Berger and Kimmel, Guide to Molecular Cloning Techniques, Methods in Enzymology, Vol. 152, Academic Press, Inc. (1987); Sambrook et al., Molecular Cloning: A Laboratory Manual, (Second Edition, Cold Spring Harbor Laboratory Press; Cold Spring Harbor, N.Y.; 1989) Vols. 1-3; Current Protocols in Molecular Biology, F. M. Ausabel et al. [Eds.], Current Protocols, a joint venture between Green Publishing Associates, Inc. and John Wiley & Sons, Inc. (supplemented through 2000); Harlow et al., Monoclonal Antibodies: A Laboratory Manual, Cold Spring Harbor Laboratory Press (1988), Paul [Ed.]; Fundamental Immunology, Lippincoft Williams & Wilkins (1998); and Harlow et al., Using Antibodies: A Laboratory Manual, Cold Spring Harbor Laboratory Press (1998).

MN-Preferential Carbonic Anhydrase Inhibitors

The novel methods of the present invention comprise inhibiting the growth of preneoplastic/neoplastic cells with compounds that preferentially inhibit the enzymatic activity of MN protein. Said compounds are organic or inorganic, preferably organic, more preferably sulfonamides. Still more preferably, said compounds are pyridinium derivatives of aromatic or heterocyclic sulfonamides. These preferred pyridinium derivatives of sulfonamides are likely to have fewer side effects than other compounds in three respects: they are small molecules, they are membrane-impermeant, and they are specific potent inhibitors of the enzymatic activity of the tumor-associated MN protein.

The pyridinium derivatives of sulfonamides useful according to the present invention can be formed, for example, by creating bonds between pyrylium salts and aromatic or heterocyclic sulfonamide reagents, as described in U.S. Patent Application No. 2004/0146955. The aromatic or heterocyclic sulfonamide portion of a pyridinium salt of a sulfonamide compound can be called the “head,” and the pyridinium portion can be called the “tail.”

It can be appreciated by those of skill in the art that various other MN-preferential carbonic anhydrase inhibitors can be useful according to the present invention.

MN Gene Therapy Vectors

Recent therapeutic strategies proposed to target aggressive tumors involve the utilization of the hypoxia-responsive promoters that can drive the expression of cytotoxic genes selectively in hypoxic tumor cells. This strategy requires that the promoter is turned on in the hypoxic conditions and turned off in the normoxic conditions. Several approaches are based on the use of repetitive hypoxia-responsive elements to achieve a higher magnitude of the hypoxic activation.

MN/CA9 is an excellent candidate for such hypoxia-regulated therapies in that it is one of the most tightly regulated by hypoxia genes, if not the most tightly regulated by hypoxia gene, found so far. However, even MN/CA9 displays some transcription activity under normoxia.

The inventors found as shown in the Examples below, that the expression of the MN/CA9 gene is subject to regulation by the MAPK kinase pathway in a HIF-1 independent manner, which may affect the transcription from the MN/CA9 promoter when employed for the therapeutic purposes, especially upon the use of multiple HRE-PR1 elements. Therefore, it may be important to use MAPK kinase pathway inhibitors in combination with MN gene therapy vectors in the treatment of MN-overexpressing diseases, for complementary therapeutic effects.

For inhibiting the expression of the MN gene using an oligonucleotide, it is possible to introduce the oligonucleotide into the targeted cell by use of gene therapy. The gene therapy can be performed by using a known method. For example, either a non-viral transfection, comprising administering the oligonucleotide directly by injection, or a transfection using a virus vector can be used, among other methods known to those of skill in the art. A preferred method for non-viral transfection comprises administering a phospholipid vesicle such as a liposome that contains the oligonucleotide, as well as a method comprising administering the oligonucleotide directly by injection. A preferred vector used for a transfection is a virus vector, more preferably a DNA virus vector such as a retrovirus vector, an adenovirus vector, an adeno-associated virus vector and a vaccinia virus vector, or a RNA virus vector.

The following examples are for purposes of illustration only and are not meant to limit the invention in any way.

### EXAMPLE 1

### Inhibition of ERK in HEK293 Cells

The inventors used PR1-HRE promoter region of CA9 (−50/+31) that contains HRE element and SP1 binding site, and assessed its transcriptional activity in luciferase-renilla reporter system. First, the PR1-HRE-luc plasmid was co-transfected together with renilla internal standard to HEK293 cells plated in high density and treated by the U0126 inhibitor of ERK (MAP kinases) for 20 h (FIG. 8). The promoter activity was analyzed in the transfected cells incubated in normoxic conditions or in the presence of DFO that can induce a chemical hypoxia. The promoter activity was determined as a ratio between the luciferase-related luminescence and renilla-related luminescence. U0126 treatment resulted in diminished PR1-HRE transcriptional activity both in normoxia and in hypoxia.

### EXAMPLE 2

### CA9 Transcriptional Activity in CGL1 and CGL3 Cells Treated with ERK Inhibitor U0126

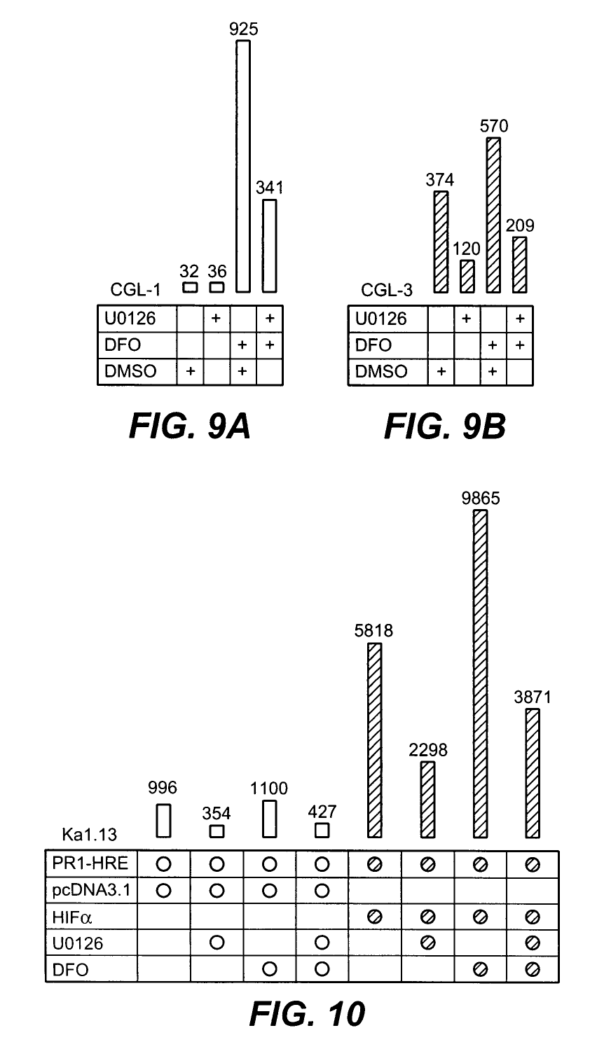

This result was corroborated in HeLa cells (not shown) and in CGL1 and CGL3 cell lines. CGL1 cells do not express CA9 in a sparse culture in normoxia, but the expression can be highly induced by hypoxia. On the other hand, CGL3 cells express CA9, but the expression level can be increased by high density as well as by hypoxia. As shown on FIG. 9, ERK inhibitor suppressed the transcription from PR1-HRE promoter region of CA9 in both cell lines treated by DFO and also in CGL3 cells plated in the high-density culture. The effect of the ERK inhibitor is evident also at the level of CA IX protein in HeLa cells [(Western blotting analysis of CA IX protein expression in sparse and dense HeLa cells, incubated in normoxia (21% O₂) and hypoxia (2% O₂), and treated by ERK inhibitor U0126 (data not shown)].

### EXAMPLE 3

### CA9 Transcriptional Activity in Ka1.13 Cells Treated with ERK Inhibitor U0126

To dissect a direct contribution of HIF-1 to transcriptional activation of CA9 promoter, the inventors performed an additional experiment using Ka1.13 cell line that is defective for HIF-1α. The Ka1.13 cells were transfected either with PR1-HRE-luc reporter plasmid+renilla standard+control pcDNA3.1 plasmid or with the PR1-HRE-luc reporter plasmid+renilla standard+HIF-1α cDNA. The results (shown in FIG. 10) revealed that inhibition of ERK led to diminished transcription from PR1-HRE promoter both in the presence and in the absence of HIF-1α. This finding indicates an important, HIF-1 independent role of ERK pathway in the control of CA9 transcription.

### EXAMPLE 4

### CA9 Transcriptional Activity with Dominant-Negative ERK Mutants in Dense HEK293 Cells

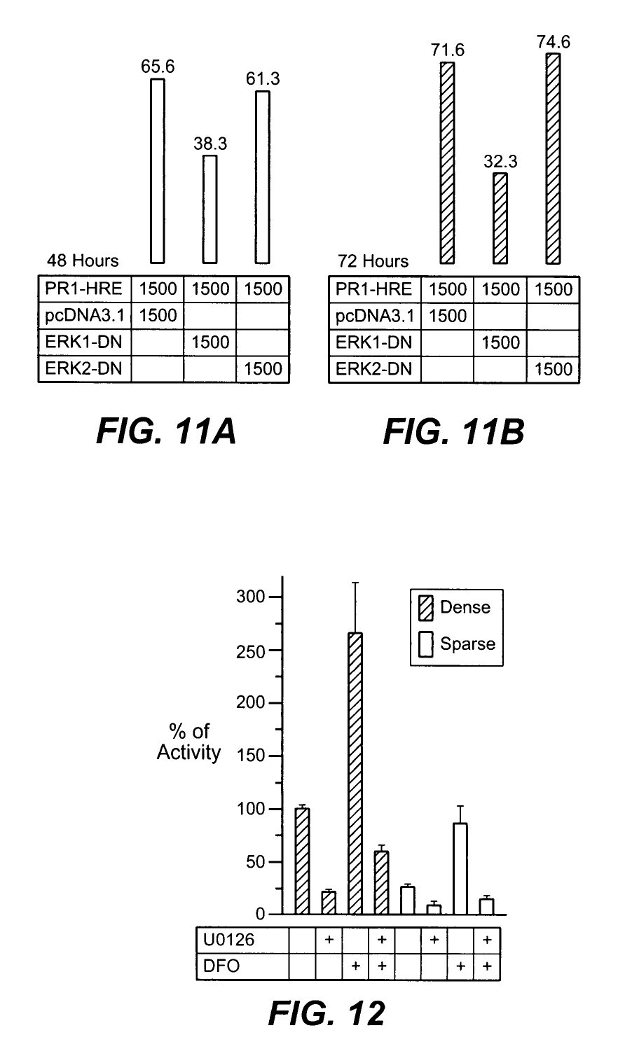

To determine which type of ERK is involved in the regulation of CA9, the inventors analyzed the promoter activity in the HEK293 cells transfected by dominant negative mutants of ERK1 and ERK2. Interestingly, the ERK1 but not ERK2 dominant-negative mutant interfered with the CA9 promoter activity. The decrease of CA9-driven transcription was observed in the normoxic cells of high density [FIG. 11] further supporting the view that ERK1 is involved in HIF-1 independent regulation of CA9.

Discussion: The inventors found that the expression of CA9 gene is subject to regulation by the MAP kinase pathway in a HIF-1 independent manner. Such regulation may affect transcription from the CA9 promoter when employed for therapeutic purposes, especially upon the use of multiple HRE-PR1 elements. That finding may also help to explain an incomplete overlap between the intratumoral distribution of HIF-1α and CA IX and should be taken into account in evaluating immunohistochemical analyses of hypoxic tumors.

### EXAMPLES 5-9

### Materials and Methods

The following Materials and Methods were used for Examples 5-9:

Cell Culture and Hypoxic Treatment

HeLa cells derived from human cervical carcinoma and HEK293 human embryonic kidney cells were cultured in DMEM supplemented with 10% FCS (BioWhittaker, Verviers, Belgium) under humidified air containing 5% CO₂ at 37° C. Ka13 derivative of CHO-K1 Chinese hamster cells (kindly provided by Dr. Patrick Maxwell, Imperial College of Science, Technology and Medicine, London, UK) [Wood et al., J. Biol. Chem., 273: 8360-8368 (1998)] were cultured in Ham's F12 medium with 10% FCS. The cells were exposed to hypoxia (1% O₂) in an anaerobic workstation (Ruskin Technologies, Bridgend, UK) in 5% CO₂, 10% H₂ and 84% N₂ at 37° C. Hypoxia was also induced chemically either with 200 μM deferoxamine mesylate (DFO, Sigma, St. Louis, Mo.), an iron chelator commonly used in the study of hypoxia-induced responses, or with 0.75 mM 2-oxoglutaratedependent dioxygenase inhibitor dimethyloxalylglycine (DMOG, Frontier Scientific, Logan Utah).

Inhibitors, Antibodies and Plasmids

The MAPK pathway inhibitor U0126 (Sigma), the PI3K inhibitor LY294002 (Calbiochem, Cambridge, Mass.) and the SP1 inhibitor mithramycin A (MMA, Sigma) were dissolved in dimethyl sulfoxide (DMSO) at 10 mM, and stored in aliquots at −20° C. Prior to use, the inhibitors were diluted in culture medium to working concentrations, i.e. 20 μM U0126, 10 μM LY294002 and 100 nM MMA. The final concentration of DMSO was less than 0.2% including controls. Cultures were pre-incubated with the inhibitors 1 h before the induction of hypoxia or addition of DFO. Cytotoxic drug effects were monitored by the colorimetric Cell Titer Blue method (Promega, Madison, Wis.).

M75 mouse monoclonal antibody specific for the N-terminal PG region of the CA IX protein was described previously [Pastorekova et al., Virology, 187: 620-626 (1992); Zavada et al., B. J. Cancer, 82: 1508-1513 (2000)]. Secondary anti-mouse antibodies conjugated with horse-radish peroxidase were purchased from Sevapharma (Prague, Czech Republic).

The PR1-HRE-luc promoter construct was generated by an insertion of a −50/+37 CA9 genomic region amplified by PCR upstream of the firefly luciferase gene in pGL3-Basic luciferase reporter vector (Promega). pRL-TK renilla vector (Promega) served for the control of the transfection efficiency. HIF-1α cDNA in pcDNA1/Neo/HIF-1α expression plasmid [Wood et al., J. Biol. Chem., 273: 8360-8368 (1998)] was kindly provided by Dr. Patrick Maxwell. Dominant-negative mutants of ERK1 (pcDNA-ERK1) and ERK2 (pcDNA-ERK2) mutated in their ATP binding sites were kindly provided by Dr. M. H. Cobb (Southwestern Medical Center, Dallas) [Minet et al., FEBS Lett., 468: 53-58 (2000)].

Transient Transfection and Luciferase Assay

The cells were plated into 30 mm Petri dishes to reach approximately 60% monolayer density on the next day. Transfection was performed with the 2 μg of PR1-HRE-luc and 100 ng of pRL-TK plasmids DNAs using a GenePorterII reagent (Gene Therapy Systems, San Diego, Calif.) according to the manufacturer's recommendation. One day later, the transfected cells were trypsinized and plated in triplicates into 24-well plates at different densities so that the dense culture contained eight times more cells than the sparse one. The cells were allowed to attach for 20 h, then they were pre-treated with inhibitors for 1 h and transferred to hypoxia (or treated with DFO) for additional 24 h. Reporter gene expression was assessed 48 h after the transfection using the Dual-Luciferase Reporter Assay System (Promega) and the luciferase activity was normalized against the renilla expression.

Immunoblotting

HeLa cells were plated in dense (80,000 cells/cm²) and sparse (10,000 cells/cm²) cultures and incubated for 24 h. Then the cells were pre-treated with inhibitors for 1 h and transferred to hypoxia for 24 h. Parallel control dishes were pre-treated and maintained in normoxia for the same time period.

For the detection of CA IX, the cells were extracted with cold RIPA buffer for 15 min at 4° C. The extracts were then centrifuged (15 min at 13,000 rpm) and total protein concentrations were determined by BCA assay (Pierce, Rockford, Ill.). Samples of 30 μg total proteins were separated by the electrophoresis using 10% SDS-PAGE and blotted onto the PVDF membrane. Before immunodetection, the membrane was treated by the blocking buffer containing 5% non-fat milk in PBS with 0.2% Nonidet P-40 for 1 h and incubated for 1 h with M75 MAb diluted 1:2 in the blocking buffer. Then the membrane was washed, incubated for 1 h with the anti-mouse secondary antibody, washed again and developed with the ECL detection system. Intensity of CA IX-specific bands was analyzed by the Scion Image Beta 4.02 software (Scion Corporation, Frederick, Md.), and relative CA IX expression was expressed as percentages.

### EXAMPLE 5

### Inhibition of the MAPK Pathway Reduces CA9 Promoter Activity and CA IX Protein Expression in Both Hypoxia and High Density

Previous studies have determined PR1-HRE as a crucial cell density- and hypoxia-inducible module within the CA9 promoter [Kaluz et al., Cancer Res., 62: 4469-4477 (2002); Kaluz et al., Cancer Res., 63: 917-922 (2003)]. Therefore, the inventors have cloned a −50/+37 CA9 genomic region, containing that module in its natural context relative to the transcription start site, upstream of the reporter luciferase gene. The PR1-HRE-luc promoter construct was then co-transfected with the renilla-coding control plasmid to HeLa cells that express CA IX protein in response to hypoxia and high cell density. In accord with earlier observations, the highest CA9 promoter activity was obtained in a dense culture exposed to the hypoxia-mimicking agent DFO. Treatment of the cells with the MAPK pathway inhibitor U0126 resulted in an about fourfold decrease of the CA9 promoter activity irrespective of the conditions used for cell incubation [FIG. 12]. CA9 promoter induction and its U0126 inhibition were comparable in physiological hypoxia (not shown).

In addition, endogenous CA IX protein levels produced in HeLa cells upon MAPK pathway inhibition under hypoxia and/or high density corresponded with the promoter activities (data not shown). Similar results were obtained when the PR1-HRE-luc construct was transfected to HEK293 cells that do not express endogenous CA IX protein, but contain the transcriptional machinery needed for the activation of the CA9 promoter by hypoxia as well as by high cell density. The increase in CA9 promoter activity observed in a dense culture exposed to hypoxia was considerably inhibited by treatment with the U0126 inhibitor of the MAPK pathway (data not shown).

### EXAMPLE 6

### Inhibition of the MAPK Pathway Reduces CA9 Promoter Activity in the Presence as Well as in the Absence of HIF-1α

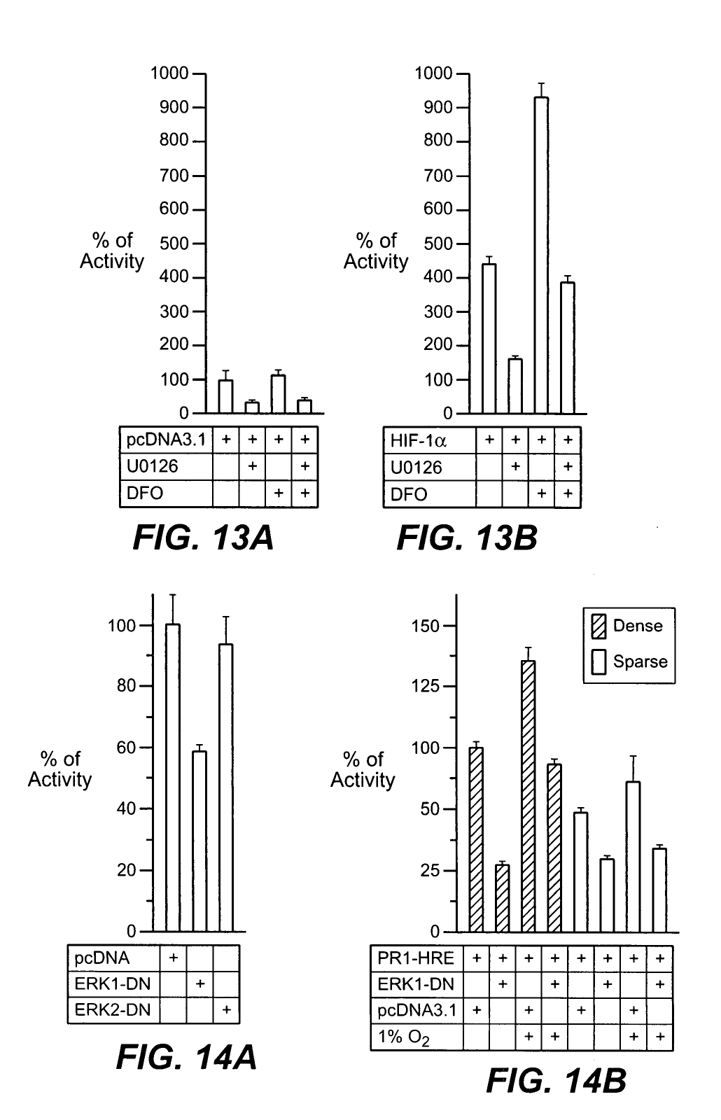

In order to determine whether regulation of CA9 expression by the MAPK pathway depends on the presence of HIF-1α, the inventors used CHO-derived Ka13 cells that do not express endogenous HIF-1α protein [Wood et al., J. Biol. Chem., 273: 8360-8368 (1998)]. Those HIF-1α-deficient cells had previously been shown to be unable to activate normally the CA9 promoter in response to cell density [Kaluz et al., Cancer Res., 62: 4469-4477 (2002)]. Therefore, the Ka13 cells were plated at intermediate density, transfected with the PR1-HRE-luc promoter construct together with the renilla control plasmid and pcDNA3.1 plasmid, pre-treated with the U0126 MAPK pathway inhibitor and exposed to a DFO-induced hypoxia. Parallel dishes were maintained in absence of DFO. The CA9 promoter activity was not increased in hypoxia apparently due to the absence of the HIF-1α protein. In spite of that lack of increase in CA9 promoter activity, inhibition of the MAPK pathway by U0126 diminished the promoter activity to less than a half in both conditions [FIG. 13A].

Co-transfection of the PR1-HRE-luc construct with a cDNA encoding the wild-type HIF-1α led to a remarkable elevation of the CA9 promoter activity, which was further increased upon DFO-induced hypoxia, possibly as a result of the stabilization of the ectopically expressed HIF-1α [FIG. 13B]. In correspondence with the results of the previous experiments, MAPK pathway inhibition reduced the promoter activity to approximately one third of a HIF-1α induced value. Those results indicate that the MAPK pathway can affect CA9 expression both via a HIF-1-mediated transcriptional activation and via a HIF-1α-independent mechanism.

### EXAMPLE 7

### CA9 Promoter Activity Decreases Upon Expression of a Dominant Negative Mutant of ERK1

MAPK pathway signaling (particularly MEK 1/2 signaling) is transmitted essentially via two downstream mediators, namely the serine/threonine kinases MAPK/ERK1 and MAPK/ERK2 [Lewis et al., Adv. Cancer Res., 74: 49-139 (1998)]. To learn which of the two ERKs is involved in the control of CA9 expression, the inventors co-transfected the PR1-HRE-luc plasmid with the plasmids encoding the dominant-negative (DN) kinase-dead mutants of either ERK1 or ERK2 into dense HEK293 cells. Those cells were chosen for the experiment due to their capacity to allow for a very high efficiency in transient co-transfection and for their full competence to drive transcription from the CA9 promoter as mentioned above.

On the other hand, HeLa cells have a high basal level of MAPK activity even in the absence of serum [Berra et al., J. Biol. Chem., 273: 10792-10797 (1998)], and transient co-transfection with DN mutants is not sufficient to get consistent results. Luciferase activities obtained in the transfected HEK293 cells and normalized against the renilla control revealed that co-expression of ERK1-DN reduced the CA9 promoter activity by about 40%, whereas co-expression of ERK2-DN had no significant effect [FIG. 14A]. Based on that finding, the inventors performed a co-transfection of PR1-HRE-luc with ERK1-DN to the cells grown in sparse and dense conditions under normoxia and hypoxia. As expected from the earlier experiments, expression of a kinase-dead mutant of ERK1 negatively affected the CA9 promoter activity in all examined conditions [FIG. 14B]. Hypoxic induction of the CA9 promoter was not as dramatic as seen before in HeLa cells, possibly due to a lower level of HIF-1α protein in HEK293 cells [Richard et al., J. Biol. Chem., 274: 32631-32637 (1999)]. Nevertheless, the findings clearly suggested that ERK1 participates in the MAPK pathway-related molecular mechanisms regulating the expression of CA9.

### EXAMPLE 8

### Simultaneous Inhibition of MAPK and PI3K Pathways has an Additive Negative Effect on CA9 Promoter Activity and CA IX Protein Expression

Comparison of the normoxic and hypoxic activities of the CA9 promoter in all HeLa, HEK293 and Ka13 cell lines has shown that the U0126-treatment did not completely abolish the induction of CA9 expression. That fact indicated that a part of the regulatory mechanisms, which transmit molecular signals generated by hypoxia and/or high cell density, remained functional. Previous studies provide evidence for the involvement of the PI3K pathway in the density-induced CA IX expression [Kaluz et al., Cancer Res., 62: 4469-4477 (2002)]. The inventors therefore anticipated that this PI3K pathway could be responsible for the CA9 promoter activity remaining after inhibition of the MAPK signaling.

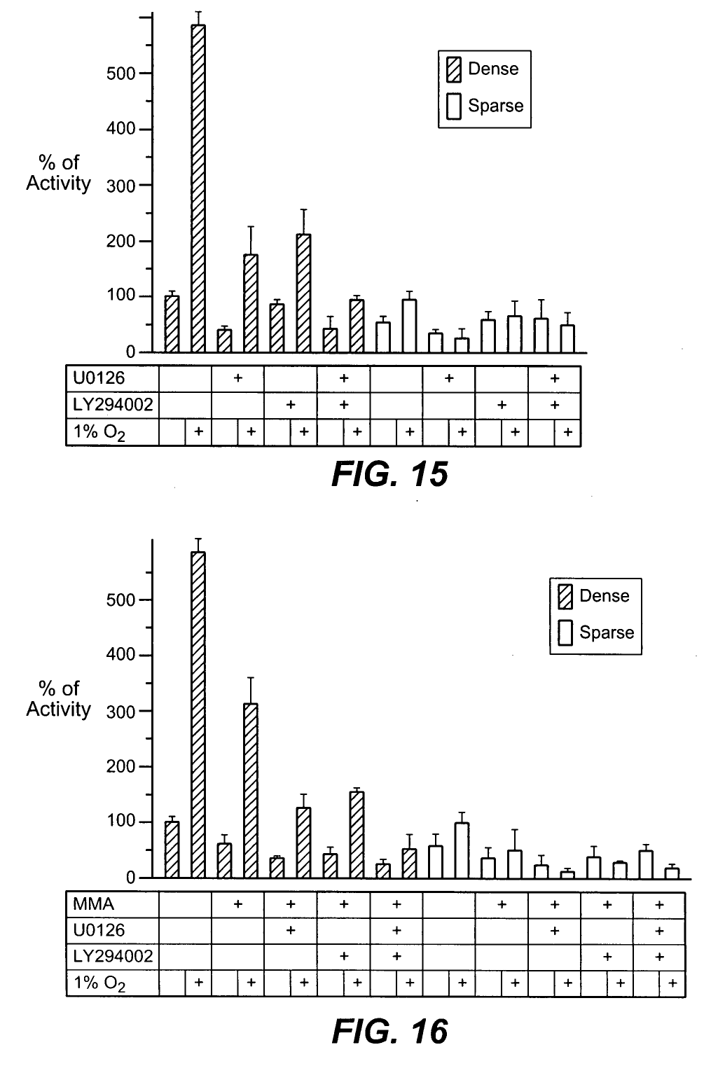

To examine that assumption, HeLa cells incubated in normoxia and physiological hypoxia were treated with inhibitors of the MAPK (U0126) and PI3K pathways (LY294002). The inhibitors were tested to determine the concentration that would give the maximum combined inhibitory effect without compromising cell survival (data not shown). Each inhibitor alone was able to reduce the CA9 promoter activity measured in dense hypoxic HeLa cells transfected with PR1-HRE-luc construct to about one third of its control value, and their simultaneous effect was still stronger [FIG. 15]. The effects of the inhibitors were less pronounced in the normoxic and sparse cells, but showed a similar tendency.

Immunoblotting analysis of endogenous CA IX protein expression in HeLa cells treated with the inhibitors confirmed that CA IX protein level was considerably diminished by the LY294002 inhibitor alone, and addition of the U0126 inhibitor caused its further decrease (data not shown). That effect could be observed under both high and low cell density. The inhibitors similarly reduced CA IX protein expression induced in HeLa cells by DMOG, a hydroxylase inhibitor that increases stability and activity of HIF-1α (data not shown). Altogether, the results allowed the inventors to conclude that both the PI3K and MAPK pathways act in parallel to activate the CA9 gene both in hypoxia and in high cell density.

### EXAMPLE 9

### Inhibition of SP1 Activity Further Reduces CA9 Gene Expression Induced by Hypoxia and/or High Cell Density

The inventors' findings presented above suggest that inhibition of the MAPK pathway interfered with a principal activating mechanism that functions under low oxygen supply as well as in the normoxic cells grown in a dense culture. The triggered signal transduction pathways seem to be integrated by a PR1-binding SP1 transcription factor, which was shown to be required for the cooperative interaction with HRE-binding HIF-1α under both conditions [Kaluz et al., Cancer Res., 63: 917-922 (2003)]. Therefore, the inventors treated PR1-HRE-luc-transfected HeLa cells with the SP1 inhibitor MMA and with the MAPK pathway (particularly MEK 1/2) inhibitor U0126. The experiment included also the PI3K inhibitor LY294002 combined with MMA.

Treatment of the transfected cells with MMA+U0126 and MMA+LY294002, respectively, resulted in stronger inhibition of CA9 promoter activity when compared to MMA alone. Simultaneous addition of all inhibitors showed an augmented effect, which was especially marked in dense hypoxic culture [FIG. 16]. Also, MMA and U0126 each separately reduced the CA IX protein level, but the combination of inhibitors almost completely prevented the hypoxic induction of CA IX protein expression (data not shown). Inhibition of HeLa cells grown in a dense culture gave very similar results.

The data confirm that SP1 mediates both hypoxia and density induced trans-activation signals as proposed by Kaluz et al. [Kaluz et al., Cancer Res., 63: 917-922 (2003)]. Kaluz et al. also indicate that CA9 gene expression accepts signals transmitted by PI3K and MAPK at least partially via SP1, and that those paths may overlap and/or complement each other in regulation of the CA9 promoter.

Discussion: The MAPK pathway plays an important role in transduction of extracellular signals exerted by various mitogenic and micro-environmental factors [Lewis et al., Adv. Cancer Res., 74: 49-139 (1998)]. Depending on the cell type and culture conditions, hypoxia and mitogenic stimulation can work together to enhance the activation of the MAPK pathway and up-regulation of HIF-1 activity. It is not surprising that the MAPK pathway significantly influences the expression of the HIF-1 targets, such as VEGF [Berra et al., Biochem. Pharmacol, 60: 1171-1178 (2000)].

Nevertheless, HIF-1α is not the only transcription factor regulated by MAPK, and HIF-1-independent mechanisms of MAPK-regulated expression of different genes including VEGF have been described [Milanini et al., J. Biol. Chem., 273: 18165-18172 (1998); Haddad, J. J., Int. Immunopharmacol., 4: 1249-1285 (2004)].

As disclosed herein, the inventors analyzed the transcriptional regulation of the CA9 gene that is a direct target of HIF-1, which binds to the HRE element adjacent to the transcription initiation site. CA9 is strongly induced by hypoxia [Wykoff et al., Cancer Res., 60: 7075-7083 (2000)]. In addition, CA9 transcription can be up-regulated under normoxic conditions by a high cell density [Lieskovska et al., Neoplasma, 46: 17-24 (1999)]. Density-induced CA9 expression involves a pericellular hypoxia, depends on subhypoxic levels of HIF-1 and is mediated by PI3K signaling [Kaluz et al., Cancer Res., 62: 4469-4477 (2002)]. PI3K is a key component of another signal transduction pathway that is activated under hypoxia, up-regulates HIF-1 by increasing protein synthesis of HIF-1α subunit and can also transmit extracellular signals in a HIF-1 independent manner [Laughner et al., Mol. Cell. Biol., 21: 3995-4004 (2001); Zhong et al., Cancer Res., 60: 1541-1545 (2000); Jiang et al., Cell Growth Differ., 12: 363-369 (2001)]. The list of targets includes VEGF that is expressed in response to activation of the PI3K pathway under hypoxia as well as under normoxia [Jiang et al., Cell Growth Differ. 12: 363-369 (2001); Stiehl et al., FEBS Lett., 512: 157-162 (2002); Jiang et al., PNAS (USA), 97: 1749-1753 (2000); Maity et al., Cancer Res., 60: 5879-5886 (2000)]. Altogether, VEGF expression is subjected to a complex regulation by at least three major signal transduction pathways driven by HIF-1, PI3K and MAPK.

Based on the principal significance of the MAPK pathway in regulation of gene expression in diverse cellular processes and using the VEGF gene as a paradigm, the inventors decided to investigate whether the MAPK pathway contributes to control of CA9 transcription. If so, they also wanted to know whether MAPK signaling is important either for hypoxic induction of CA9 expression or for its up-regulation by density, or for both. Therefore, the inventors analyzed the activity of a crucial PR1-HRE portion of the CA9 promoter in cells grown in low and high densities, as well as in those maintained in low and normal oxygen levels. The cells transfected by the PR1-HRE-luc promoter construct were re-plated to different densities, pre-treated with the MAPK pathway (particularly MEK 1/2) U0126 inhibitor and then subjected to hypoxia. In both HeLa and HEK293 cell lines used, the inventors observed a remarkable reduction of the CA9 promoter activity following inhibition of the MAPK pathway in all tested conditions of cell incubation. The same expression pattern was obtained with an endogenous CA IX protein expressed in the MAPK inhibitor-treated HeLa cells. The results have shown that the MAPK pathway is actually involved in the control of CA9 gene expression both in hypoxia and high cell density.

Of course, effects of the MAPK pathway inhibition could rely on HIF-1, as mentioned above, and was also shown for the PI3K pathway in the density-induced CA9 expression [Kaluz et al., Cancer Res., 62: 4469-4477 (2002)]. Indeed, involvement of HIF-1 was indirectly supported by the finding of a negative regulation of the CA9 promoter activity by a dominant-negative mutant of MAPK/ERK1, but not ERK2, since only ERK1 can phosphorylate and activate HIF-1α [Minet et al., FEBS Lett., 468: 53-58 (2000)]. However, examination of the CA9 promoter activity in HIF-1α deficient Ka13 cells and in the same cells co-transfected with the HIF-1α cDNA revealed that at least a part of the response to MAPK inhibition is not dependent on HIF-1α. In the absence of HIF-1α, Ka13 cells are unable to induce CA9 expression in hypoxia, and a high cell density has only a weak stimulatory effect [Kaluz et al., Cancer Res., 62: 4469-4477 (2002)], but the treatment with the MAPK inhibitor can lower even the basal CA9 promoter activity to less than half. Upon ectopic expression of HIF-1α, CA9 activity markedly increases, and inhibition of the MAPK pathway brings it back down to about one third of the control level. Thus, both HIF-1-dependent and independent components appear to be involved in the transmission of regulatory signals by the MAPK pathway to the CA9 promoter.

Interestingly, the MAPK-inhibited cells still retain the capacity to induce CA9 transcription in hypoxia and in high cell density, so additional regulators are apparently involved. In fact, PI3K was already proven to play a role in CA9 control in a dense culture and also seems to participate in HIF-1-mediated induction of CA9 in hypoxia, because its inhibition leads to a decreased level of HIF-L a protein and consequently to a diminished level of CA IX protein in HeLa cells [Kaluz et al., Cancer Res., 62: 4469-4477 (2002)]. Indeed, simultaneous treatment of the dense and hypoxic HeLa cells with the MAPK and PI3K inhibitors had an augmented negative effect on the CA9 promoter activity as well as on CA IX protein expression.

The resulting promoter activity and level of the CA IX protein were very low or even absent, clearly indicating that the pathways complement each other in the control of CA9 gene, and that they are responsible for a significant part of density-induced as well as hypoxia-induced CA9 expression.

Previous dissection of the transcriptional factors that execute the hypoxic and density-generated signals by direct binding to the CA9 promoter revealed HIF-1 and SP1 as key players and demonstrated that SP1 activity is required for the full transcription of the CA9 gene under both conditions [Kaluz et al., Cancer Res., 63: 917-922 (2003)]. Whereas SP1 functioning is obligatory for CA9 induction by density, it seemed to be needed only for an improvement of HIF-1-mediated CA9 transcription under hypoxia [Kaluz et al., Cancer Res., 63: 917-922 (2003)]. In the present experiments, inhibition of SP1 considerably reduced the hypoxia-induced CA IX protein expression. That effect was stronger after additional inhibition of the MAPK pathway, suggesting that the MAPK pathway, which is constitutively activated in HeLa cells [Berra et al., J. Biol. Chem., 273: 10792-10797 (1998)], cooperates with SP1 in proper signal transduction to the CA9 promoter. The same explanation possibly applies to SP1's role in MAPK and/or PI3K mediated CA9 transcription in high cell density. Based on these data, SP1 clearly behaves as an important component of the basal CA9 transcription machinery, which is required for the full performance of both the MAPK and PI3K pathways. That fits well with the current view of SP1 as an acceptor and integrator of signals from the two pathways upon their activation by hypoxia and by extracellular factors [Berra et al., Biochem. Pharmacol., 60: 1171-1178 (2000); Jiang et al., J. Biol. Chem., 278: 31964-31967 (2003); Huang et al., World J. Gastroenterol., 10: 809-812 (2004)].

Taken together, in this work the inventors provided the evidence for an involvement of the MAPK pathway in the regulation of CA9 expression. They demonstrated that the MAPK pathway controls the CA9 promoter via both HIF-1-dependent and HIF-1 independent signals, and that it works along with the PI3K pathway and SP1 as a downstream mediator of CA9 transcriptional responses to both hypoxia and high cell density. That is an important finding, since activating mutations of various components of both MAPK and PI3K pathways, which occur in many tumor types [Vogelstein and Kinzler, Nat. Med., 10: 789-799 (2004)], may up-regulate CA9 gene expression inside as well as outside of the hypoxic regions and influence an intratumoral distribution of the CA IX protein. CA IX is functionally implicated in tumor growth and survival [Svastova et al., Exp. Cell Res., 290: 332-345 (2003); Svastova et al., FEBS Lett., 577: 439-445 (2004); Robertson et al., Cancer Res., 64: 6160-6165 (2004)], and its increased expression may thus have important consequences for tumor biology.

### ATCC DEPOSITS

The materials listed below were deposited with the American Type Culture Collection (ATCC) now at 10810 University Blvd., Manassus, Va. 20110-2209 (USA). The deposits were made under the provisions of the Budapest Treaty on the International Recognition of Deposited Microorganisms for the Purposes of Patent Procedure and Regulations thereunder (Budapest Treaty). Maintenance of a viable culture is assured for thirty years from the date of deposit. The hybridomas and plasmids will be made available by the ATCC under the terms of the Budapest Treaty, and subject to an agreement between the Applicants and the ATCC which assures unrestricted availability of the deposited hybridomas and plasmids to the public upon the granting of patent from the instant application. Availability of the deposits is not to be construed as a license to practice the invention in contravention of the rights granted under the authority of any Government in accordance with its patent laws.

| Hybridoma | Deposit Date | ATCC # |
|---|---|---|
| VU-M75 | Sep. 17, 1992 | HB 11128 |
| MN 12.2.2 | Jun. 9, 1994 | HB 11647 |

| Plasmid | Deposit Date | ATCC # |
|---|---|---|
| A4a | Jun. 6, 1995 | 97199 |
| XE1 | Jun. 6, 1995 | 97200 |
| XE3 | Jun. 6, 1995 | 97198 |

Similarly, the hybridoma cell line V/10-VU which produces the V/10 monoclonal antibodies was deposited on Feb. 19, 2003 under the Budapest Treaty at the International Depository Authority (IDA) of the Belgian Coordinated Collections of Microorganisms (BCCM) at the Laboratorium voor Moleculaire Biologie-Plasmidencollectie (LMBP) at the Universeit Gent, K. L. Ledeganckstraat 35, B-9000 Gent, Belgium [BCCM/LMBP] under the Accession No. LMBP 6009CB.

The description of the foregoing embodiments of the invention have been presented for purposes of illustration and description. They are not intended to be exhaustive or to limit the invention to the precise form disclosed, and obviously many modifications and variations are possible in light of the above teachings. The embodiments were chosen and described in order to explain the principles of the invention and its practical application to enable thereby others skilled in the art to utilize the invention in various embodiments and with various modifications as are suited to the particular use contemplated.

All references cited herein are hereby incorporated by reference.

1. A method of treating a mammal for a preneoplastic/neoplastic disease, wherein said disease is characterized by abnormal MN/CA9 gene expression, comprising administering to said mammal a therapeutically effective amount of a composition comprising a MAPK pathway inhibitor.

2. The method of claim 1, wherein said MAPK pathway inhibitor is a raf kinase inhibitor.

3. The method of claim 2, wherein said raf kinase inhibitor is the bis aryl-urea Sorafenib (BAY 43-9006) or an omega-carboxypyridyl substituted urea.

4. The method of claim 2, wherein said raf kinase inhibitor is the bis aryl-urea Sorafenib (BAY 43-9006).

5. The method of claim 1, wherein said MAPK pathway inhibitor is conjugated to an antibody or biologically active antibody fragment which specifically binds MN/CA IX.

6. The method of claim 1 further comprising administering to said mammal radiation and/or a therapeutically effective amount in a physiologically acceptable formulation of one or more of the following compounds selected from the group consisting of: conventional anticancer drugs, chemotherapeutic agents, different inhibitors of cancer-related pathways, bioreductive drugs, gene therapy vectors, CA IX-specific antibodies and CA IX-specific antibody fragments that are biologically active.

7. The method of claim 6, wherein said inhibitors of cancer-related pathways are inhibitors of the PI3K pathway.

8. The method of claim 6, wherein said gene therapy vectors are targeted to hypoxic tumors.

9. The method of claim 1, wherein said preneoplastic/neoplastic disease characterized by abnormal MN/CA9 gene expression is selected from the group consisting of mammary, urinary tract, bladder, kidney, ovarian, uterine, cervical, endometrial, squamous cell, adenosquamous cell, vaginal, vulval, prostate, liver, lung, skin, thyroid, pancreatic, testicular, brain, head and neck, mesodermal, sarcomal, stomach, spleen, gastrointestinal, esophageal, and colon preneoplastic/neoplastic diseases.

10. The method of claim 1 wherein said disease is a normoxic tumor.

11. The method of claim 1 wherein said disease is a hypoxic tumor.

12. The method of claim 1, wherein said mammal is a human.

13. A method of therapy selection for a human patient with a preneoplastic/neoplastic disease, comprising:
(a) detecting and quantifying the level of MN/CA9 gene expression in a sample taken from the patient; and
(b) deciding to use MAPK pathway-directed therapy to treat the patient based upon abnormal levels of MN/CA9 gene expression in the patient's sample.

14. The method of claim 13, wherein said preneoplastic/neoplastic sample is a formalin-fixed, paraffin-embedded tissue sample or a frozen tissue sample.

15. The method of claim 13, wherein said detecting and quantifying step (a) comprises immunologically detecting and quantifying the level of MN/CA IX protein in said sample.

16. The method according to claim 15, wherein said immunologically detecting and quantifying comprises the use of an assay selected from the group consisting of Western blots, enzyme-linked immunosorbent assays, radioimmunoassay, competition immunoassays, dual antibody sandwich assays, immunohistochemical staining assays, agglutination assays, and fluorescent immunoassays.

17. The method according to claim 15, wherein said immunologically detecting and quantifying comprises the use of the monoclonal antibody secreted by the hybridoma VU-M75 which has Accession No. ATCC HB 11128.

18. The method of claim 13 wherein said MAPK-directed therapy is a raf kinase inhibitor.

19. The method of claim 18 wherein said raf kinase inhibitor is the bis aryl-urea Sorafenib (BAY 43-9006) or an omega-carboxypyridyl substituted urea.

20. The method of claim 18 wherein said raf kinase inhibitor is the bis aryl-urea Sorafenib (BAY 43-9006).

21. The method of claim 13 wherein said MAPK pathway inhibitor is conjugated to an antibody or a biologically active antibody fragment which specifically binds MN/CA IX.

22. The method of claim 13 wherein said MAPK pathway inhibitor is administered in combination with one or more additional therapies.

23. The method of claim 22, wherein said one or more additional therapies target MN/CA9 gene expression or MN/CA IX enzymatic activity.
